(19) United States
(12) Patent Application Publication
(10) Pub. No.: US 2016/0069883 A1
(43) Pub. Date: Mar. 10, 2016

(54) BIOMARKERS FOR DETECTION OF BREAST CANCER

(71) Applicant: Provista Diagnostics Inc., New York, NY (US)

(72) Inventors: David E. Reese, New York, NY (US); Rao V. Mulpuri, Apex, NC (US); Meredith C. Henderson, Mesa, AZ (US); Kasey Lee Benson, Scottsdale, AZ (US)

(21) Appl. No.: 14/846,563

(22) Filed: Sep. 4, 2015

Related U.S. Application Data
(60) Provisional application No. 62/046,042, filed on Sep. 4, 2014.

(51) Int. Cl. G01N 33/574; C12Q 1/68

(52) U.S. Cl. CPC G01N 33/57415; C12Q 1/6886; C12Q 2600/158; C12Q 2600/16; C12Q 2600/178

(57) ABSTRACT

Methods are provided for predicting and diagnosing the presence of breast cancer, as well as for assessing the therapeutic efficacy of a cancer treatment and determining whether a subject potentially is developing cancer.

### CROSS REFERENCE TO RELATED APPLICATION(S)

This application claims the benefit under 35 USC §119(e) of U.S. Application Ser. No. 62/046,042, filed Sep. 4, 2014, the entire content of which is incorporated herein by reference.

### BACKGROUND

1. Field of Invention

The present invention relates generally to methods for cancer detection, and more particularly to methods for predicting and diagnosing breast cancer.

2.Background Information

There is intense effort in the search for biomarkers that can detect early disease and/or monitor for disease progression and recurrence. With the advent of molecularly-targeted therapeutics, biomarkers that are associated with biological subtypes of cancer may be useful for predicting responses to therapeutic interventions.

Protein-based, approaches to distinguish cancer-bearing patient sera from healthy control sera have been challenged by the difficulty in identifying small quantities of protein fragments within complex protein mixtures, protein instability, and natural variations in protein content within patient populations. Autoantibodies (AAb) to tumor antigens have advantages as potential cancer biomarkers as they are stable, highly specific, easily purified from serum, and are readily detected with well-validated secondary reagents. Although they have high specificities to distinguish cancer from control sera, most tumor-associated autoantibodies (TAAbs) demonstrate poor sensitivities. Testing multiple antigens in parallel may serve to increase the predictive value of tumor-specific antibodies for use as immunodiagnostics.

There are several platforms that may be utilized to screen for immune responses using tumor antigens. For example, protein microarrays offer a platform to present tumor antigens to screen for immune responses. Protein microarrays are capable of presenting and assessing hundreds of tumor antigens simultaneously. The responses are rapidly identified because the address of each protein is known in advance and there are no representation issues; all proteins, even rare ones, are represented equally (usually in duplicate). The proteins are arrayed on a single microscope slide requiring only a few microliters of serum per assay. Known tumor antigens as well as predicted tumor antigens can be included to generate a comprehensive protein tumor antigen array.

A major need in the precise diagnosis of cancer is the use of complementary technologies to existing standard of care such as imaging and patient exam. A biochemical tool that could aid in the correct identification of breast cancer lesions in conjunction with imaging would provide the physician a real-time evaluation mechanism for both high risk and screening patients. Indeed, a major controversy in annual screening for breast cancer is a high rate of over-diagnosis. At the same time, imaging is still the predominant technology used to detect breast cancers. It is therefore advantageous to contemplate an improvement of existing standard of care (reduction of false positives and false negatives) utilizing a combination of proteomic and imaging approaches in the detection of breast cancer.

### SUMMARY OF THE INVENTION

The present invention generally relates to cancer biomarkers and particularly to biomarkers associated with breast cancer. It provides methods to predict, evaluate, diagnose, and monitor cancer, particularly breast cancer, by measuring certain biomarkers. A set of biomarkers including serum protein biomarkers (SPBs) and TAAbs provides a detectable molecular signature of breast cancer in a subject.

Accordingly, in one embodiment, the invention provides a method for determining whether a subject has or is at risk of having breast cancer. The method includes obtaining a biological sample from the subject; and measuring a level of at least one autoantibody in the sample and at least one protein biomarker, both as compared with a healthy subject's sample; wherein a level of antibody and biomarker greater than that found in the healthy sample, is indicative of a subject having or at risk of having breast cancer.

In another embodiment, the method includes: a) obtaining a biological sample from the subject; b) measuring a level of at least one protein biomarker and at least one autoantibody; c) determining whether the level is elevated; and d) providing a determination of whether the subject has or is at risk of having breast cancer. In embodiments, the protein biomarker is one or more proteins selected from FasL, TNFA, IL8, CEA, ERBB2, HGF, IFNG, IL6, OPN, VEGFC, VEGFD, ATF3, ATP6AP1, BDNF, CTBP1, DBT, EIF3E, FRS3, HOXD1, p53, PDCD6IP, RAC3, SELL, SF3A1, SOX2, TFCP2, TRIMP2, UBAP1, ZMYM6, IGF2PB2, MUC1, BAT4, BMX, C15orf48, CSNK1E, GPR157, MYOZ2, RAB5A, SERPINH1, SLC33A1 and ZNF510.

In another embodiment, the invention provides a method for measuring the level of a protein biomarker and an autoantibody in a sample from a subject having or at risk of having breast cancer. The method includes: a) obtaining a biological sample from the subject; and b) measuring a level of at least one protein biomarker and at least one autoantibody, wherein the protein biomarker is selected from FasL, TNFA, IL8, CEA, ERBB2, HGF, IFNG, IL6, OPN, VEGFC, VEGFD, ATF3, ATP6AP1, BDNF, CTBP1, DBT, EIF3E, FRS3, HOXD1, p53, PDCD6IP, RAC3, SELL, SF3A1, SOX2, TFCP2, TRIMP2, UBAP1, ZMYM6, IGF2PB2, MUC1, BAT4, BMX, C15orf48, CSNK1E, GPR157, MYOZ2, RAB5A, SERPINH1, SLC33A1, ZNF510, and combinations thereof.

In embodiments, the level of the at least one protein biomarker is determined by protein array analysis.

In yet another embodiment, the present invention provides a kit for detecting breast cancer in a subject. The kit includes means for detecting in a biological sample at least one protein biomarker and at least one autoantibody.

In another embodiment, the present invention provides an array comprising a plurality of probes for specifically binding a biomarker or autoantibody. The probes may include oligonucleotides or polypeptides.

A panel of biomarkers for use with the invention includes the following proteins and fragments thereof: FasL, TNFA, IL8, CEA, ERBB2, HGF, IFNG, IL6, OPN, VEGFC, VEGFD, ATF3, ATP6AP1, BDNF, CTBP1, DBT, EIF3E, FRS3, HOXD1, p53, PDCD6IP, RAC3, SELL, SF3A1, SOX2, TFCP2, TRIMP2, UBAP1, ZMYM6, IGF2PB2, MUC1, BAT4, BMX, C15orf48, CSNK1E, GPR157, MYOZ2, RAB5A, SERPINH1, SLC33A1 and ZNF510. In embodiments, the panel and method include one or more autoantibodies that specifically bind one or more of RAC3, IGF2BP2, MUC1, ErbB2, ATP6AP1, PDCD6IP, DBT, CSNK1E, FRS3, HOXD1, SF3A1, CTBP1, C15orf48, MYOZ2, EIF3E, BAT4, ATF3, BMX, RAB5A, UBAP1, SOX2, GPR157, BDNF, ZMYM6, SLC33A1, TRIM32, ALG10, TFCP2, SERPINH1, SELL, ZNF510 or p53. In embodiments, the method utilizes one or more p53TAABs.

### BRIEF DESCRIPTION OF THE DRAWINGS

FIG. 1 is a pictorial representation illustrating the concept of blood-based protein biomarker detection when combined with standard imaging.

FIG. 2 is a pictorial representation illustrating that detection of autoantibodies (AAb) or serum protein biomarkers (SPB) depends on the protein production for the tumor, tumor microenvironment and host-tumor responses.

FIG. 3 is a table presenting experimental data relating to characteristics of the patient population used to test whether SPBs and AAbs improve prediction of breast cancer.

FIG. 4 is a series of graphical representation presenting experimental data. The Figure presents box plots depicting SPB concentrations for selected biomarkers in benign and cancer groups in 163 patients. Upper left=FasL, Upper right=TNFA, Lower left=IL8, and Lower right=CEA.

FIG. 5 is a tabular list of AAbs used in analysis of contribution to sensitivity/specificity by this class of biomarker in embodiments of the invention.

FIG. 6 is a table presenting experimental data relating to characteristics of the patient population used to test whether SPBs and AAbs improve prediction of breast cancer.

FIG. 7 is a table presenting experimental data relating to comparison of models using SPB alone and SPB in combination with AAbs.

FIG. 8 is a graphical representation presenting experimental data. The Figure presents a box plot depicting SPB concentration for a selected biomarker (CEA) in benign and cancer groups in 351 patients.

FIG. 9 is a graphical representation presenting experimental data. The Figure presents a box plot depicting SPB concentration for a selected biomarker (ERBB2) in benign and cancer groups in 351 patients.

FIG. 10 is a graphical representation presenting experimental data. The Figure presents a box plot depicting SPB concentration for a selected biomarker (FASL) in benign and cancer groups in 351 patients.

FIG. 11 is a graphical representation presenting experimental data. The Figure presents a box plot depicting SPB concentration for a selected biomarker (HGF) in benign and cancer groups in 351 patients.

FIG. 12 is a graphical representation presenting experimental data. The Figure presents a box plot depicting SPB concentration for a selected biomarker (IFNG) in benign and cancer groups in 351 patients.

FIG. 13 is a graphical representation presenting experimental data. The Figure presents a box plot depicting SPB concentration for a selected biomarker (IL6) in benign and cancer groups in 351 patients.

FIG. 14 is a graphical representation presenting experimental data. The Figure presents a box plot depicting SPB concentration for a selected biomarker (IL8) in benign and cancer groups in 351 patients.

FIG. 15 is a graphical representation presenting experimental data. The Figure presents a box plot depicting SPB concentration for a selected biomarker (OPN) in benign and cancer groups in 351 patients.

FIG. 16 is a graphical representation presenting experimental data. The Figure presents a box plot depicting SPB concentration for a selected biomarker (TNFA) in benign and cancer groups in 351 patients.

FIG. 17 is a graphical representation presenting experimental data. The Figure presents a box plot depicting SPB concentration for a selected biomarker (VEGFC) in benign and cancer groups in 351 patients.

FIG. 18 is a graphical representation presenting experimental data. The Figure presents a box plot depicting SPB concentration for a selected biomarker (VEGFD) in benign and cancer groups in 351 patients.

FIG. 19 is a graphical representation presenting experimental data. The Figure presents a box plot depicting SPB concentration for a selected biomarker (ATF3) in benign and cancer groups in 351 patients.

FIG. 20 is a graphical representation presenting experimental data. The Figure presents a box plot depicting SPB concentration for a selected biomarker (ATP6AP1) in benign and cancer groups in 351 patients.

FIG. 21 is a graphical representation presenting experimental data. The Figure presents a box plot depicting SPB concentration for a selected biomarker (BDNF) in benign and cancer groups in 351 patients.

FIG. 22 is a graphical representation presenting experimental data. The Figure presents a box plot depicting SPB concentration for a selected biomarker (CTBP1) in benign and cancer groups in 351 patients.

FIG. 23 is a graphical representation presenting experimental data. The Figure presents a box plot depicting SPB concentration for a selected biomarker (DBT) in benign and cancer groups in 351 patients.

FIG. 24 is a graphical representation presenting experimental data. The Figure presents a box plot depicting SPB concentration for a selected biomarker (EIF3E) in benign and cancer groups in 351 patients.

FIG. 25 is a graphical representation presenting experimental data. The Figure presents a box plot depicting SPB concentration for a selected biomarker (FRS3) in benign and cancer groups in 351 patients.

FIG. 26 is a graphical representation presenting experimental data. The Figure presents a box plot depicting SPB concentration for a selected biomarker (HOXD1) in benign and cancer groups in 351 patients.

FIG. 27 is a graphical representation presenting experimental data. The Figure presents a box plot depicting SPB concentration for a selected biomarker (p53) in benign and cancer groups in 351 patients.

FIG. 28 is a graphical representation presenting experimental data. The Figure presents a box plot depicting SPB concentration for a selected biomarker (PDCD6IP) in benign and cancer groups in 351 patients.

FIG. 29 is a graphical representation presenting experimental data. The Figure presents a box plot depicting SPB concentration for a selected biomarker (RAC3) in benign and cancer groups in 351 patients.

FIG. 30 is a graphical representation presenting experimental data. The Figure presents a box plot depicting SPB concentration for a selected biomarker (SELL) in benign and cancer groups in 351 patients.

FIG. 31 is a graphical representation presenting experimental data. The Figure presents a box plot depicting SPB concentration for a selected biomarker (SF3A1) in benign and cancer groups in 351 patients.

FIG. 32 is a graphical representation presenting experimental data. The Figure presents a box plot depicting SPB concentration for a selected biomarker (50X2) in benign and cancer groups in 351 patients.

FIG. 33 is a graphical representation presenting experimental data. The Figure presents a box plot depicting SPB concentration for a selected biomarker (TFCP2) in benign and cancer groups in 351 patients.

FIG. 34 is a graphical representation presenting experimental data. The Figure presents a box plot depicting SPB concentration for a selected biomarker (TRIM32) in benign and cancer groups in 351 patients.

FIG. 35 is a graphical representation presenting experimental data. The Figure presents a box plot depicting SPB concentration for a selected biomarker (UBAP1) in benign and cancer groups in 351 patients.

FIG. 36 is a graphical representation presenting experimental data. The Figure presents a box plot depicting SPB concentration for a selected biomarker (ZMYM6) in benign and cancer groups in 351 patients.

FIG. 37 is a graphical representation presenting experimental data. The Figure presents a box plot depicting SPB concentration for a selected biomarker (IGF2PB2) in benign and cancer groups in 351 patients.

FIG. 38 is a graphical representation presenting experimental data. The Figure presents a box plot depicting SPB concentration for a selected biomarker (MUC1) in benign and cancer groups in 351 patients.

FIG. 39 is a graphical representation presenting experimental data. The Figure presents a box plot depicting SPB concentration for a selected biomarker (BAT4) in benign and cancer groups in 351 patients.

FIG. 40 is a graphical representation presenting experimental data. The Figure presents a box plot depicting SPB concentration for a selected biomarker (BMX) in benign and cancer groups in 351 patients.

FIG. 41 is a graphical representation presenting experimental data. The Figure presents a box plot depicting SPB concentration for a selected biomarker (C15orf48) in benign and cancer groups in 351 patients.

FIG. 42 is a graphical representation presenting experimental data. The Figure presents a box plot depicting SPB concentration for a selected biomarker (CSNK1E) in benign and cancer groups in 351 patients.

FIG. 43 is a graphical representation presenting experimental data. The Figure presents a box plot depicting SPB concentration for a selected biomarker (GPR157) in benign and cancer groups in 351 patients.

FIG. 44 is a graphical representation presenting experimental data. The Figure presents a box plot depicting SPB concentration for a selected biomarker (MYOZ2) in benign and cancer groups in 351 patients.

FIG. 45 is a graphical representation presenting experimental data. The Figure presents a box plot depicting SPB concentration for a selected biomarker (RAB5A) in benign and cancer groups in 351 patients.

FIG. 46 is a graphical representation presenting experimental data. The Figure presents a box plot depicting SPB concentration for a selected biomarker (SERPINH1) in benign and cancer groups in 351 patients.

FIG. 47 is a graphical representation presenting experimental data. The Figure presents a box plot depicting SPB concentration for a selected biomarker (SLC33A1) in benign and cancer groups in 351 patients.

FIG. 48 is a graphical representation presenting experimental data. The Figure presents a box plot depicting SPB concentration for a selected biomarker (ZNF510) in benign and cancer groups in 351 patients.

FIG. 49 is a graphical representation presenting experimental data. The Figure presents a box plot depicting SPB concentration for a selected biomarker (ErbB2) in benign and cancer groups in 351 patients.

FIG. 50 is a graphical representation presenting experimental data. The Figure presents a box plot depicting SPB concentration for a selected biomarker (ErbB2) in benign and cancer groups in 351 patients.

FIG. 51 is a graphical representation presenting experimental data. The Figure presents a box plot depicting SPB concentration for a selected biomarker (MUC1) in benign and cancer groups in 351 patients.

FIG. 52 is a graphical representation presenting experimental data. The Figure presents a box plot depicting SPB concentration for a selected biomarker (MUC1) in benign and cancer groups in 351 patients.

FIG. 53 is a tabular list of AAbs used in analysis of contribution to sensitivity/specificity by this class of biomarker in embodiments of the invention.

FIG. 54 is a graphical representation presenting experimental data. The Figure presents a plot depicting sensitivity/specificity (greater than 90%) of cancer detection in patients utilizing detection of SPB of the invention in combination with detection of TAAbs of the invention.

FIG. 55 is a graphical representation presenting experimental data. The Figure presents a plot depicting sensitivity/specificity (less than 72%) of cancer detection in patients utilizing detection of SPB of the invention alone.

FIG. 56 is a graphical representation presenting experimental data. The Figure presents a plot depicting sensitivity/specificity (less than 84%) of cancer detection in patients utilizing detection of TAAbs of the invention alone.

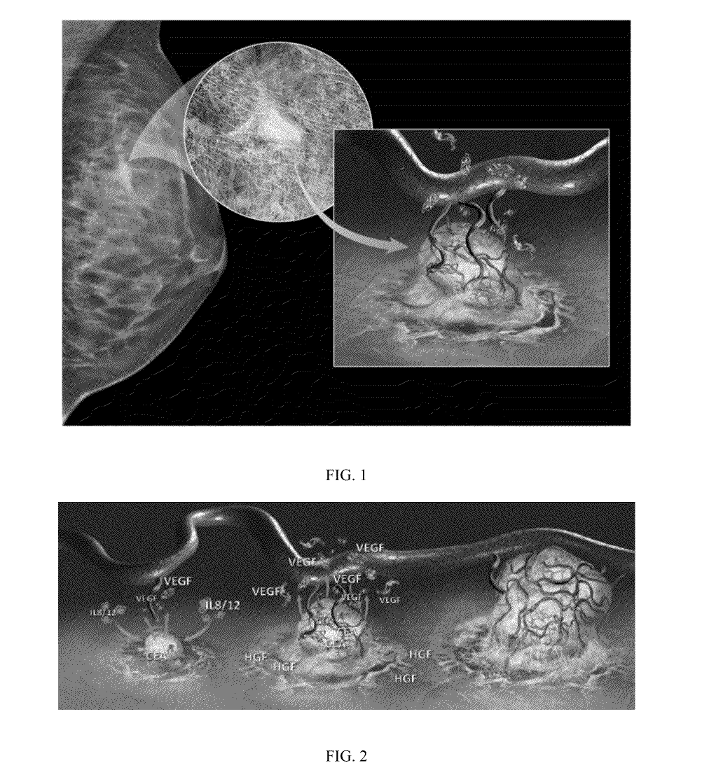

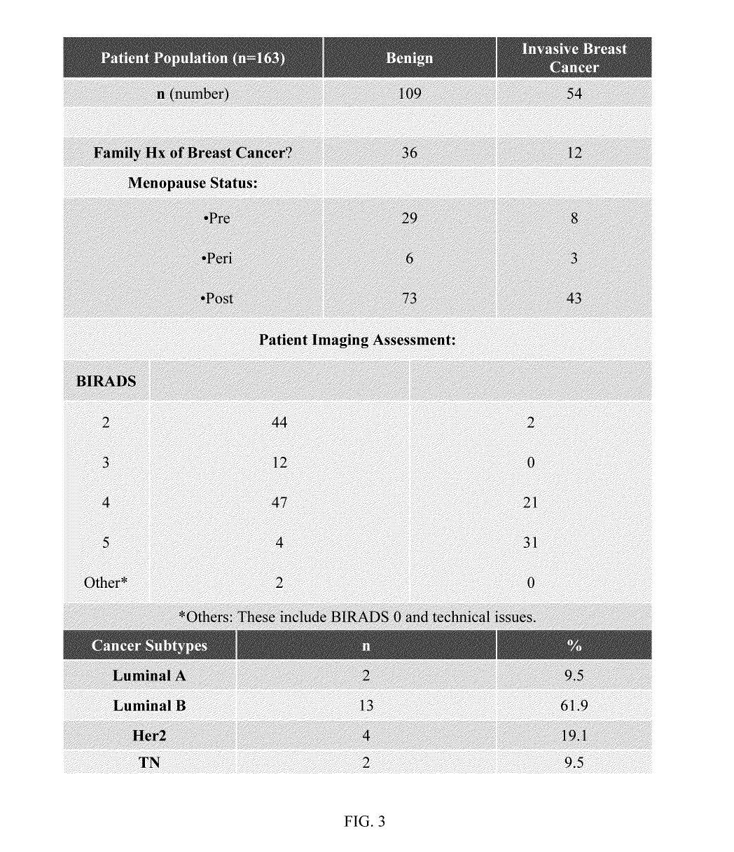

### DETAILED DESCRIPTION OF THE INVENTION

The present invention relates to biornarkers associated with breast cancer. It provides methods to predict, evaluate, diagnose, and monitor cancer, particularly breast cancer, by measuring certain biomarkers. A set of biomarkers including serum protein biomarkers and TAAbs provides a detectable molecular signature of breast cancer in a subject. Further, the present application evidences the proof of concept that AAbs and SPB combined provide greater sensitivity and specificity to differentiate benign and breast cancer than either biomarker alone.

Before the present compositions and methods are further described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The presently disclosed subject matter provides a panel of biomarkers including proteins, specifically serum proteins, in combination with TAAbs, that are useful for the detection, desirably early detection, of breast cancer. The panel of biomarkers provided herein addresses certain limitations of early detection of tumors by other methods of screening alone.

Several proteins were assessed. In various embodiments, the panel includes one or more of the following proteins as well as fragments thereof: FasL, TNFA, IL8, CEA, ERBB2, HGF, IFNG, IL6, OPN, VEGFC, VEGFD, ATF3, ATP6AP1, BDNF, CTBP1, DBT, EIF3E, FRS3, HOXD1, p53, PDCD6IP, RAC3, SELL, SF3A1, SOX2, TFCP2, TRIMP2, UBAP1, ZMYM6, IGF2PB2, MUC1, BAT4, BMX, C15orf48, CSNK1E, GPR157, MYOZ2, RAB5A, SERPINH1, SLC33A1 and ZNF510.

In combination with protein detection, the presently disclosed methodology utilizes detection of TAAbs, such as one or more TAAbs, each TAAB being specific for RAC3, IGF2BP2, MUC1, ErbB2, ATP6AP1, PDCD6IP, DBT, CSNK1E, FRS3, HOXD1, SF3A1, CTBP1, C15orf48, MYOZ2, EIF3E, BAT4, ATF3, BMX, RAB5A, UBAP1, SOX2, GPR157, BDNF, ZMYM6, SLC33A1, TRIM32, ALG10, TFCP2, SERPINH1, SELL, ZNF510 or p53. Additionally, multiple TAAbs may be utilized, wherein each of the multiple TAABs is specific for only one of RAC3, IGF2BP2, MUC1, ErbB2, ATP6AP1, PDCD6IP, DBT, CSNK1E, FRS3, HOXD1, SF3A1, CTBP1, C15orf48, MYOZ2, EIF3E, BAT4, A11:3, BMX, RAB5A, UBAP1, SOX2, GPR157, BDNF, ZMYM6, SLC33A1, TRIM32, ALG10, TFCP2, SERPINH1, SELL, ZNF510 or p53. In embodiments, multiple p53 TAAbs may be utilized, for example, up to 12 or more p53 TAAbs may be utilized.

In various embodiments detection of TAAbs may be performed using any isoform or variant of RAC3, IGF2BP2, MUC1, ErbB2, ATP6AP1, PDCD6IP, DBT, CSNK1E, FRS3, HOXD1, SF3A1, CTBP1, C15orf48, MYOZ2, EIF3E, BAT4, ATF3, BMX, RAB5A, UBAP1, SOX2, GPR157, BDNF, ZMYM6, SLC33A1, TRIM32, ALG10, TFCP2, SERPINH1, SELL, ZNF510 or p53, including wild-type, mutant, as well as protein fragments thereof.

In combination, the presently disclosed biomarkers provide significant clinical utility for the early detection of breast cancer. Accordingly, in some embodiments methods are provided for assigning a subject to a group having a higher or lower probability of breast cancer. In one embodiment, the method includes determining the level of each of a panel of biomarkers in a sample from the patient, wherein the panel comprises at least one of FasL, TNFA, IL8, and CEA, and at least one TAAbs, such as at least one p53 TAAB, and assigning the patient to the group having a higher or lower probability of breast cancer based on the determined amount of each biomarker in the panel.

In some embodiments, a method is provided for assigning a subject to a high-risk group for breast cancer.

In some embodiments, a method is provided for managing treatment of a subject with potential breast cancer.

In various embodiments, the method of the present invention provides a sensitivity/specificity greater than use of SPBs or TAAbs alone. For example, the method of the present invention provides a sensitivity/specificity of detection greater than about 85, 86, 87, 88, 89, 90, 91, 92, 93, 94, 95, 96, 97, 98 or 99% utilizing SPBs in combination with AAbs.

The level of each of the presently disclosed panel of biomarkers can be determined in a variety of animal tissues. In some embodiments, the biomarkers can be detected in samples from a subject, which include bodily fluids such as, but not limited to, serum, blood, blood plasma, urine, sputum, seminal fluid, cerebrospinal fluid, ascites, feces, lymph or nipple aspirate, breast tissue and the like.

In some embodiments, the presently disclosed methods can comprise statistically analyzing the amounts of each biomarker. The statistical analysis can comprise applying a predetermined algorithm to the amounts of the biomarkers. The results of the algorithm can be employed to assign a subject to a group having a higher or lower likelihood of breast cancer.

A “biomarker” in the context of the present invention is a molecular indicator of a specific biological property; a biochemical feature or facet that can be used to measure the progress of disease or the effects of treatment. “Biomarker” encompasses, without limitation, serum proteins and TAAbs, including their polymorphisms, mutations, variants, modifications, subunits, fragments, complexes, unique epitopes, and degradation products.

The term “polypeptide” is used in its broadest sense to refer to a polymer of subunit amino acids, amino acid analogs, or peptidomimetics, including proteins and peptoids. The polypeptides may be naturally occurring full length proteins or fragments thereof, processed forms of naturally occurring polypeptides (such as by enzymatic digestion), chemically synthesized polypeptides, or recombinantly expressed polypeptides. The polypeptides may comprise D-and/or L-amino acids, as well as any other synthetic amino acid subunit, and may contain any other type of suitable modification, including but not limited to peptidornimetic bonds and reduced peptide bonds.

In one embodiment, the disclosed methodology utilizes detection of one or more RAC3 TAAb, one or more IGF2BP2 TAAb, one or more MUC1 TAAb, one or more ErbB2 TAAb, ATP6AP1 TAAb, one or more PDCD6IP TAAb, one or more DBT TAAb, one or more CSNK1E TAAb, one or more FRS3 TAAb, one or more HOXD1 TAAb, one or more SF3A1 TAAb, one or more CTBP1 TAAb, one or more C15orf48 TAAb, one or more MYOZ2 TAAb, one or more EIF3E TAAb, one or more BAT4 TAAb, one or more ATF3 TAAb, one or more BMX TAAb, one or more RAB5A TAAb, one or more UBAP1 TAAb, one or more SOX2 TAAb, one or more GPR157 TAAb, one or more BDNF TAAb, one or more ZMYM6 TAAb, one or more SLC33A1 TAAb, one or more TRIM32 TAAb, one or more ALG10 TAAb, one or more TFCP2 TAAb, one or more SERPINH1 TAAb, one or more SELL TAAb, one or more ZNF510 TAAb, or one or more p53 TAAb. As such, the method may utilize detection of various antibodies that bind different “antigenic fragments” of RAC3, IGF2BP2, MUC1, ErbB2, ATP6AP1, PDCD6IP, DBT, CSNK1E, FRS3, HOXD1, SF3A1, CTBP1, C15orf48, MYOZ2, EIF3E, BAT4, ATF3, BMX, RAB5A, UBAP1, SOX2, GPR157, BDNF, ZMYM6, SLC33A1, TRIM32, ALG10, TFCP2, SERPINH1, SELL, ZNF510 or p53, or a variant or mutant of RAC3, IGF2BP2, MUC1, ErbB2, ATP6AP1, PDCD6IP, DBT, CSNK1E, FRS3, HOXD1, SF3A1, CTBP1, C15orf48, MYOZ2, EIF3E, BAT4, ATF3, BMX, RAB5A, UBAP1, SOX2, GPR157, BDNF, ZMYM6, SLC33A1, TRIM32, ALG10, TFCP2, SERPINH1, SELL, ZNF510 or p53. As used herein, an “antigenic fragment”” is any portion of at least 4 amino acids of a polypeptide that can give rise to an immune response. In various embodiments, an antigenic fragment is at least 5, 6, 7, 8, 9, 10, 11, 12, 13, 14, 151, 16, 17, 18, 19, 20, 21, 22, 23, 24, 25, 26, 27, 28, 29, 30, 35, 40, 45, 50, 75, 100, 150, 200, 250, 300, or the full amino acid sequence of a given polypeptide.

A variety of algorithms can be employed in the presently disclosed methods. The algorithms employed are not limited to those described herein, but rather include algorithms as would be apparent to those of ordinary skill in the art upon a review of the instant disclosure.

The level of each of a panel of biomarkers can be determined in the presently disclosed method. In some embodiments, the panel of biomarkers can comprise one or more serum proteins and at least one or more TAAbs. However, the presently disclosed subject matter is not limited to the panel of biomarkers described above. Any marker that correlates with breast cancer or the progression of breast cancer can be included in the biomarker panel provided herein, and is within the scope of the presently disclosed subject matter. Any suitable method can be utilized to identify additional breast cancer biomarkers suitable for use in the presently disclosed methods. For example, biomarkers that are known or identified as being up or down-regulated in breast cancer using methods known to those of ordinary skill in the art can be employed. Additional biomarkers can include one or more of polypeptides, small molecule metabolites, lipids and nucleotide sequences. Markers for inclusion on a panel can be selected by screening for their predictive value using any suitable method, including but not limited to, those described.

As is apparent from the foregoing embodiments, the presently disclosed method is useful for screening patients for breast cancer, for the early detection of breast cancer, and for managing the treatment of patients with potential breast cancer or with known breast cancer. For example, in some embodiments, the panel of biomarkers can be useful for screening patients prior to imaging or other known methods for detecting breast tumors, to define patients at high risk or higher risk for breast cancer. Further, the presently disclosed method may be utilized in combination with other screening methods, such as imaging or histological analysis.

In one embodiment, the presence of any amount of biomarker in a sample from a subject at risk of breast cancer can indicate a likelihood of breast cancer in the subject. In another embodiment, if biomarkers are present in a sample from a subject at risk of breast cancer, at levels which are higher than that of a control sample (a sample from a subject who does not have breast cancer) than the subject at risk of breast cancer has a likelihood of breast cancer. Subjects with a likelihood of breast cancer can then be tested for the actual presence of breast cancer using standard diagnostic techniques known to the skilled artisan, including biopsy, histological analysis or imaging, such as MM. In various embodiments, the method results in an accurate diagnosis in at least 70% of cases; more preferably of at least 75%, 80%, 85%, 90%, or more of the cases.

Any suitable method can be employed for determining the level of each of the panel of biomarkers, as would be apparent to one skilled in the art upon a review of the present disclosure. For example, a method for detecting TAAbs may include use of biomolecules immobilized on a solid support or substrate. In one embodiment, Nucleic Acid Protein Programmable Array (NAPPA) technology can be used. NAPPA arrays are generated by printing full-length cDNAs encoding the target proteins at each feature of the array. The proteins are then transcribed and translated by a cell-free system and immobilized in situ using epitope tags fused to the proteins. Other suitable immobilization methods include, but are not limited to luciferase immunoprecipitation systems (LIPS), Luminex™ beads, mass spectrophotometer, standard immune dipstick assays, standard plate-based ELISA assays, microbead-based ELISA assays.

As used herein, an array may be any arrangement or disposition of the polypeptides. In one embodiment, the polypeptides are at specific and identifiable locations on the array. Those of skill in the art will recognize that many such permutations of the polypeptides on the array are possible. In another non-limiting embodiment, each distinct location on the array comprises a distinct polypeptide.

Any suitable support or surface may be used. Examples of such supports include, but are not limited to, microarrays, beads, columns, optical fibers, wipes, nitrocellulose, nylon, glass, quartz, diazotized membranes (paper or nylon), silicones, polyformaldehyde, cellulose, cellulose acetate, paper, ceramics, metals, metalloids, semiconductive materials, coated beads, magnetic particles; plastics such as polyethylene, polypropylene, and polystyrene; and gel-forming materials, such as proteins (e.g., gelatins), lipopolysaccharides, silicates, agarose, polyacrylamides, methylmethracrylate polymers; sol gels; porous polymer hydrogels; nanostructured surfaces; nanotubes (such as carbon nanotubes), and nanoparticles (such as gold nanoparticles or quantum dots).

In one embodiment, the support is a solid support. Any suitable “solid support” may be used to which the polypeptides can be attached including but not limited to dextrans, hydrogels, silicon, quartz, other piezoelectric materials such as langasite, nitrocellulose, nylon, glass, diazotized membranes (paper or nylon), polyformaldehyde, cellulose, cellulose acetate, paper, ceramics, metals, metalloids, semiconductive materials, coated beads, magnetic particles; plastics such as polyethylene, polypropylene, and polystyrene; and gel-forming materials, such as proteins (e.g., gelatins), lipopolysaccharides, silicates, agarose and polyacrylamides.

A variety of detection techniques are also suitable for detection of serum proteins. For example, methods for detecting proteins can include gas chromatography (GC), liquid chromatography/mass spectroscopy (LC-MS), gas chromatography/mass spectroscopy (GC-MS), nuclear magnetic resonance (NMR), magnetic resonance imaging (MRI), Fourier Transform InfraRed (FT-IR), and inductively coupled plasma mass spectrometry (ICP-MS). It is further understood that mass spectrometry techniques include, but are not limited to, the use of magnetic-sector and double focusing instruments, transmission quadrapole instruments, quadrupole ion-trap instruments, time-of-flight instruments (TOF), Fourier transform ion cyclotron resonance instruments (FT-MS), and matrix-assisted laser desorption/ionization time-of-flight mass spectrometry (MALDI-TOF MS).

In some embodiments, protein biomarkers can be detected using technologies well known to those of skill in the art such as gel electrophoresis, immunohistochemistry, and antibody binding. Methods for generating antibodies against a polypeptide of interest are well known to those of ordinary skill in the art. An antibody against a protein biomarker of the presently disclosed subject matter can be any monoclonal or polyclonal antibody, so long as it suitably recognizes the protein biomarker. In some embodiments, antibodies are produced using the protein biomarker as the immunogen according to any conventional antibody or antiserum preparation process. The presently disclosed subject matter provides for the use of both monoclonal and polyclonal antibodies. In addition, a protein used herein as the immunogen is not limited to any particular type of immunogen. For example, fragments of the protein biomarkers of the presently disclosed subject matter can be used as immunogens. The fragments can be obtained by any method including, but not limited to, expressing a fragment of the gene encoding the protein, enzymatic processing of the protein, chemical synthesis, and the like.

Antibodies of the presently disclosed subject matter can be useful for detecting the protein biomarkers. For example, antibody binding is detected by techniques known in the art (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, and the like), complement fixation assays, immunofluorescence assays, protein A assays, and immunoelectrophoresis assays, and the like. Upon review of the present disclosure, those skilled in the art will be familiar with numerous specific immunoassay formats and variations thereof that can be useful for carrying out the methods of the presently disclosed subject matter.

In any embodiment of the invention, detection techniques may utilize a detectable tag, such as a detectable moiety. A tag may be linked to a polypeptide through covalent bonding, including, but not limited to, disulfide bonding, hydrogen bonding, electrostatic bonding, recombinant fusion and conformational bonding. Alternatively, a tag may be linked to a polypeptide by means of one or more linking compounds. Techniques for conjugating tags to polypeptides are well known to the skilled artisan. Detectable tags can be used diagnostically to, for example, assess the presence of antibodies, or antibodies to a protein in a sample; and thereby detect the presence of breast cancer, or monitor the development or progression of breast cancer as part of a clinical testing procedure. Any suitable detection tag can be used, including but not limited to enzymes, prosthetic groups, fluorescent materials, luminescent materials, bioluminescent materials, radioactive materials, positron emitting metals, and nonradioactive paramagnetic metal ions. The tag used will depend on the specific detection/analysis/diagnosis techniques and/or methods used such as immunohistochemical staining of (tissue) samples, flow cytometric detection, scanning laser cytometric detection, fluorescent immunoassays, enzyme-linked immunosorbent assays (ELISAs), radioimmunoassays (RIAs), bioassays (e.g., neutralization assays), Western blotting applications, and the like. For immunohistochemical staining of tissue samples preferred tags are enzymes that catalyze production and local deposition of a detectable product. Enzymes typically conjugated to polypeptides to permit their immunohistochemical visualization are well known and include, but are not limited to, acetylcholinesterase, alkaline phosphatase, beta-galactosidase, glucose oxidase, horseradish peroxidase, and urease. Typical substrates for production and deposition of visually detectable products are also well known to the skilled person in the art. The polypeptides can be labeled using colloidal gold or they can be labeled with radioisotopes.

Gene expression levels may be determined in a disclosed method using any technique known in the art. Exemplary techniques include, for example, methods based on hybridization analysis of polynucleotides (e.g., genomic nucleic acid sequences and/or transcripts (e.g., mRNA)), methods based on sequencing of polynucleotides, methods based on detecting proteins (e.g., immunohistochemistry and proteomics-based methods).

The assays described herein can be adapted to be performed by lay users without a laboratory. The users may be health care professionals in point-of-care facilities or lay consumers in field conditions. The devices may have multiple embodiments including single-use devices, simple reusable devices and computerized biomonitors. The single-use devices, similar to over-the-counter lateral flow assays for pregnancy, enable subjective multi-biomarker assays to be performed. Simple reusable devices also enable objective biomarker assays that provide a refined or enhanced indication of solid state cancer mass, and may also enable remote data processing.

Gene expression levels also can be determined by quantification of a microRNA or gene transcript (e.g., mRNA). Commonly used methods known in the art for the quantification of mRNA expression in a sample include, without limitation, northern blotting and in situ hybridization; RNAse protection assays; and PCR-based methods, such as reverse transcription polymerase chain reaction (RT-PCR) and real time quantitative PCR (also referred to as qRT-PCR). Alternatively, antibodies may be employed that can recognize specific duplexes, including DNA duplexes, RNA duplexes, and DNA-RNA hybrid duplexes, or DNA-protein duplexes. Representative methods for sequencing-based gene expression analysis include Serial Analysis of Gene Expression (SAGE), and gene expression analysis by massively parallel signature sequencing (MPSS).

Some method embodiments involving the determination of mRNA levels utilize RNA (e.g., total RNA) isolated from a target sample, such a breast cancer tissue sample. General methods for RNA (e.g., total RNA) isolation are well known in the art and are disclosed in standard textbooks of molecular biology.

Differential gene expression also can be determined using microarray techniques. In these methods, specific binding partners, such as probes (including eDNAs or oligonucleotides) specific for RNAs of interest or antibodies specific for proteins of interest are plated, or arrayed, on a microchip substrate. The microarray is contacted with a sample containing one or more targets (e.g., microRNA, mRNA or protein) for one or more of the specific binding partners on the microarray. The arrayed specific binding partners form specific detectable interactions (e.g., hybridized or specifically bind to) their cognate targets in the sample of interest.

In some examples, differential gene expression is determined using in situ hybridization techniques, such as fluorescence in situ hybridization (FISH) or chromogen in situ hybridization (CISH). In these methods, specific binding partners, such as probes labeled with a fluorophore or chromogen specific for a target cDNA, microRNA or mRNA (e.g., a biomarker cDNA or mRNA molecule or microRNA molecule) is contacted with a sample, such as a breast cancer sample mounted on a substrate (e.g., glass slide). The specific binding partners form specific detectable interactions (e.g., hybridized to) their cognate targets in the sample. For example, hybridization between the probes and the target nucleic acid can be detected, for example by detecting a label associated with the probe. In some examples, microscopy, such as fluorescence microscopy, is used.

Another aspect of the present invention is that the assay can be provided in a kit which allows for more convenient laboratory-based biomarker analysis. The kits may include a plurality of components including reagents, supplies, written instructions, and/or software. The kits may have a plurality of embodiments including laboratory kits and mail-in kits. The kits can include secondary reagents. Secondary reagents may be antibodies, enzymes, labels, or chemicals and may enable a complete biomarker panel assay.

Exemplary kits can include at least one means for detection of one or more of the disclosed panel constituents (such as, at least two, at least three, at least four, or at least five detection means). In some examples, such kits can further include at least one means for detection of one or more (e.g., one to three) housekeeping genes or proteins. Detection means can include, without limitation, a nucleic acid probe specific for a genomic sequence including a disclosed gene, a nucleic acid probe specific for a transcript (e.g., mRNA) encoded by a disclosed gene, a pair of primers for specific amplification of a disclose gene (e.g., genomic sequence or cDNA sequence of such gene), an antibody or antibody fragment specific for a protein encoded by a disclosed gene.

In some kit embodiments, the primary detection means (e.g., nucleic acid probe, nucleic acid primer, or antibody) can be directly labeled, e.g., with a fluorophore, chromophore, or enzyme capable of producing a detectable product (such as alkaline phosphates, horseradish peroxidase and others commonly known in the art). Other kit embodiments will include secondary detection means; such as secondary antibodies (e.g., goat anti-rabbit antibodies, rabbit anti-mouse antibodies, anti-hapten antibodies) or non-antibody hapten-binding molecules (e.g., avidin or streptavidin). In some such instances, the secondary detection means will be directly labeled with a detectable moiety. In other instances, the secondary (or higher order) antibody will be conjugated to a hapten (such as biotin, DNP, and/or FITC), which is detectable by a detectably labeled cognate hapten binding molecule (e.g., streptavidin (SA) horseradish peroxidase, SA alkaline phosphatase, and/or SA QDot™). Some kit embodiments may include colorimetric reagents (e.g., DAB, and/or AEC) in suitable containers to be used in concert with primary or secondary (or higher order) detection means (e.g., antibodies) that are labeled with enzymes for the development of such colorimetric reagents.

In some embodiments, a kit includes positive or negative control samples, such as a cell line or tissue known to express or not express a particular biomarker.

In some embodiments, a kit includes instructional materials disclosing, for example, means of use of a probe or antibody that specifically binds a disclosed gene or its expression product (e.g., microRNA, mRNA or protein), or means of use for a particular primer or probe. The instructional materials may be written, in an electronic form (e.g., computer diskette or compact disk) or may be visual (e.g., video files). The kits may also include additional components to facilitate the particular application for which the kit is designed. Thus, for example, the kit can include buffers and other reagents routinely used for the practice of a particular disclosed method. Such kits and appropriate contents are well known to those of skill in the art.

Certain kit embodiments can include a carrier means, such as a box, a bag, a satchel, plastic carton (such as molded plastic or other clear packaging), wrapper (such as, a sealed or sealable plastic, paper, or metallic wrapper), or other container. In some examples, kit components will be enclosed in a single packaging unit, such as a box or other container, which packaging unit may have compartments into which one or more components of the kit can be placed. In other examples, a kit includes a one or more containers, for instance vials, tubes, and the like that can retain, for example, one or more biological samples to be tested.

Other kit embodiments include, for instance, syringes, cotton swabs, or latex gloves, which may be useful for handling, collecting and/or processing a biological sample. Kits may also optionally contain implements useful for moving a biological sample from one location to another, including, for example, droppers, syringes, and the like. Still other kit embodiments may include disposal means for discarding used or no longer needed items (such as subject samples). Such disposal means can include, without limitation, containers that are capable of containing leakage from discarded materials, such as plastic, metal or other impermeable bags, boxes or containers.

The kits can further include software. Software may include a training video that may provide additional support including demonstration of biomarker assays, examples of results, or educational materials for performing biomarker assays according to the invention.

The following examples are provided to further illustrate the embodiments of the present invention, but are not intended to limit the scope of the invention. While they are typical of those that might be used, other procedures, methodologies, or techniques known to those skilled in the art may alternatively be used.

### EXAMPLE 1

### Detection of Breast Cancer

This example demonstrates the proof of concept that AAbs and SPBs combined provide greater sensitivity and specificity to differentiate benign and breast cancer than either biomarker alone.

SPBs in over 163 serum samples from women with either breast cancer or benign breast conditions were measured. 10 SPBs and 28 AAbs associated with breast cancer, were analyzed in 31 of these patients.

Methods and Population

163 serum samples were obtained from Mercy Women's Hospital for a prospectively collected set of patients who were being evaluated for a suspicious mass. A SPB training model using MSD based instrumentation was developed to determine protein concentrations and develop predictive ranges for women with benign and invasive lesions.

Results

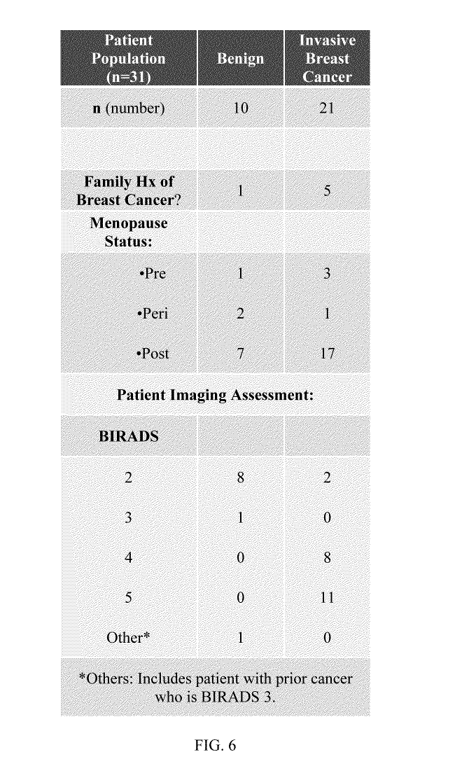

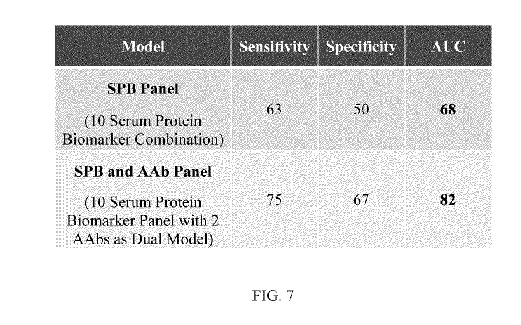

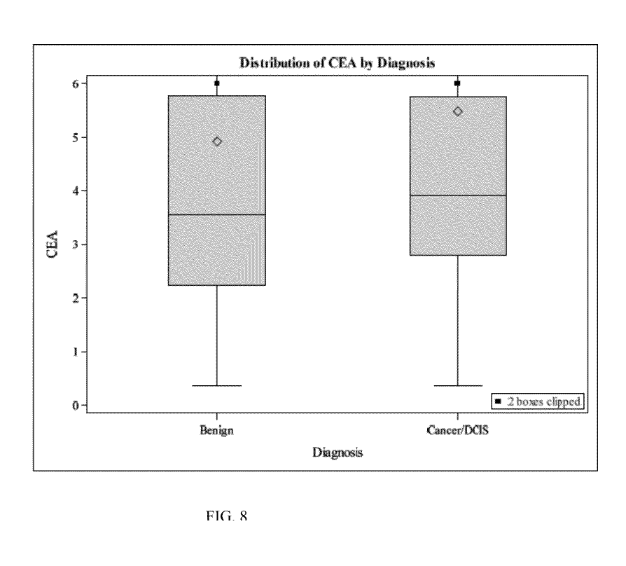

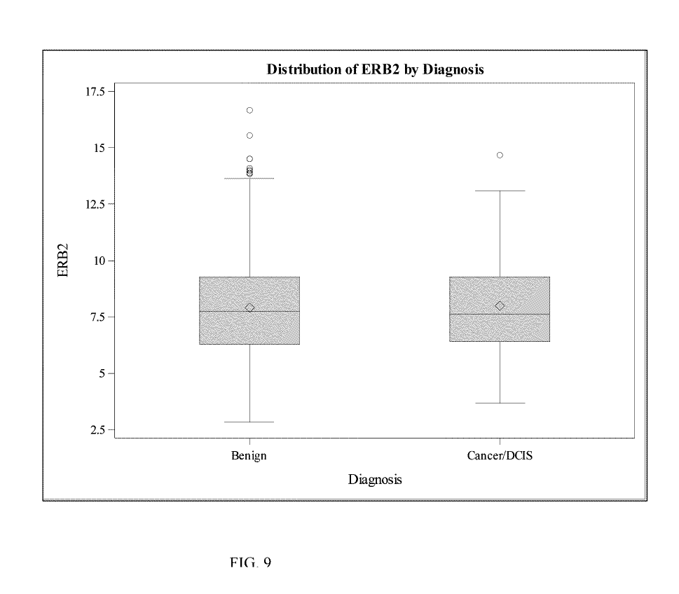

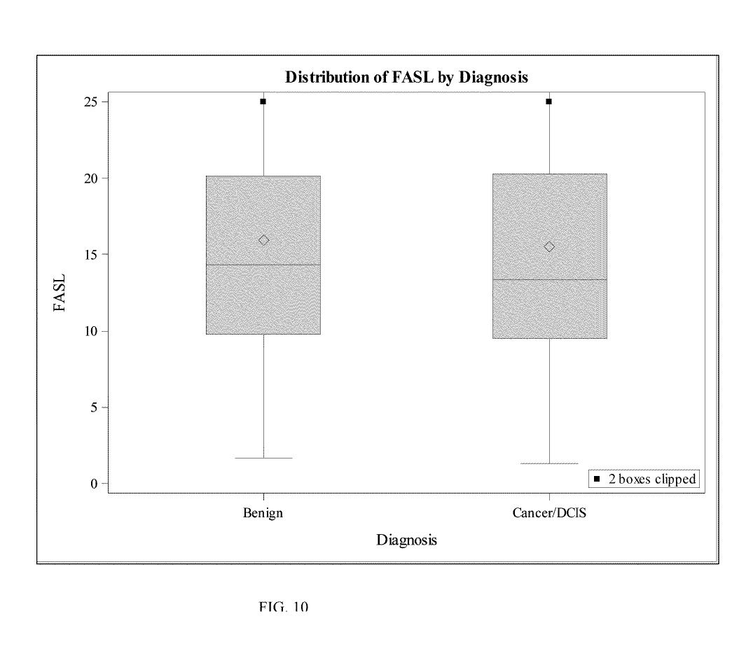

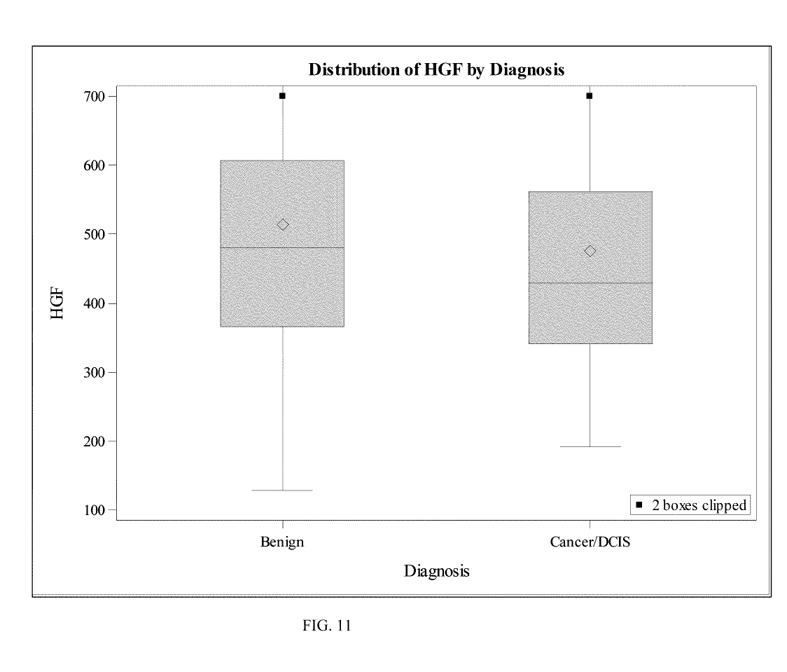

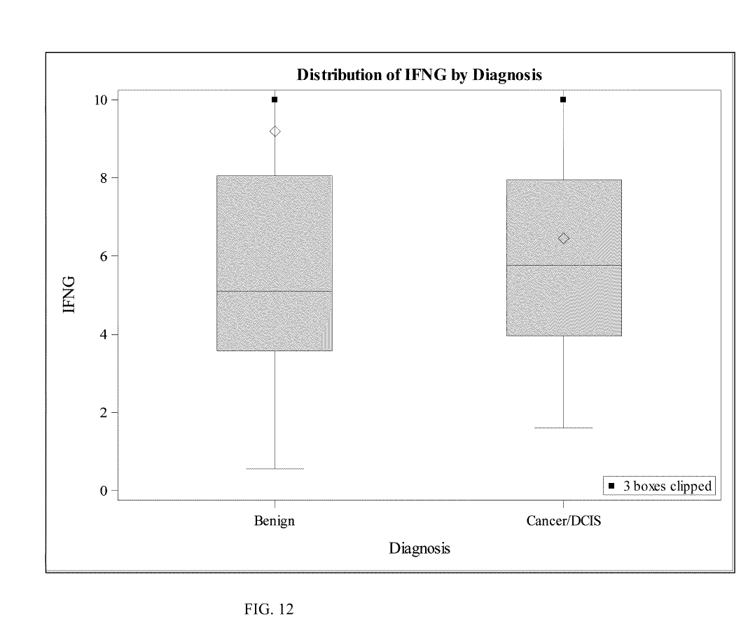

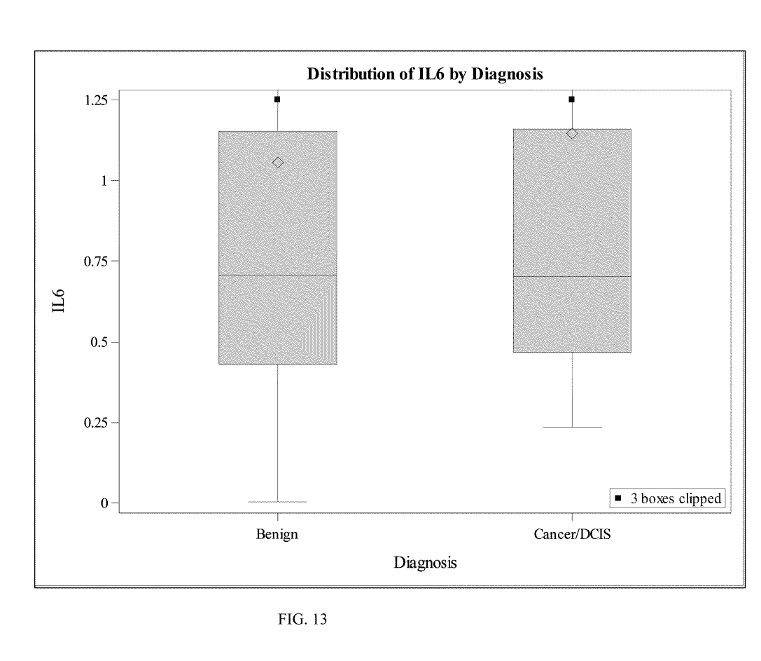

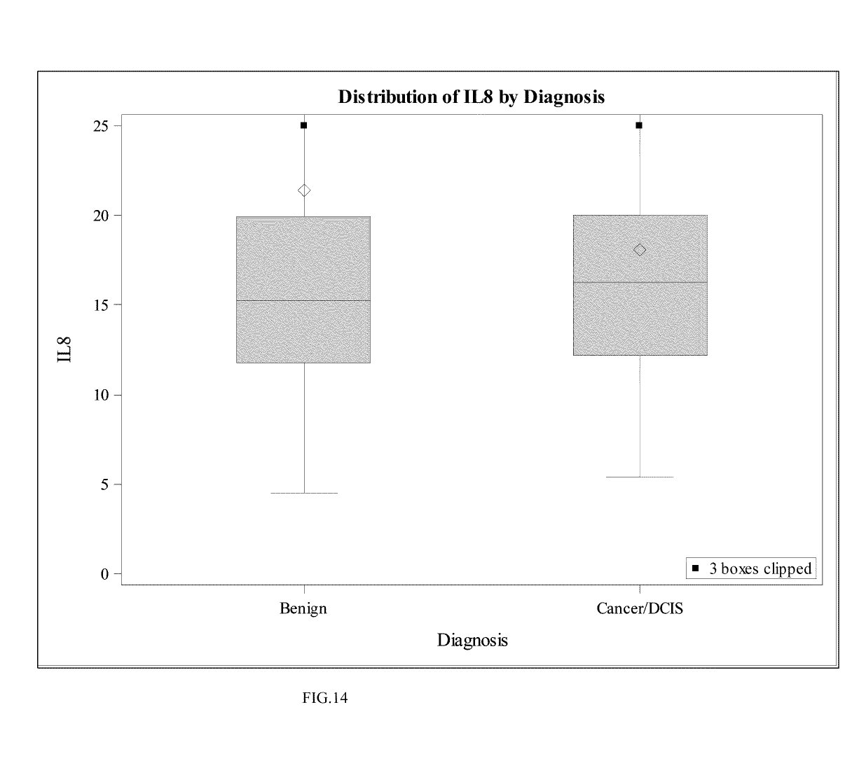

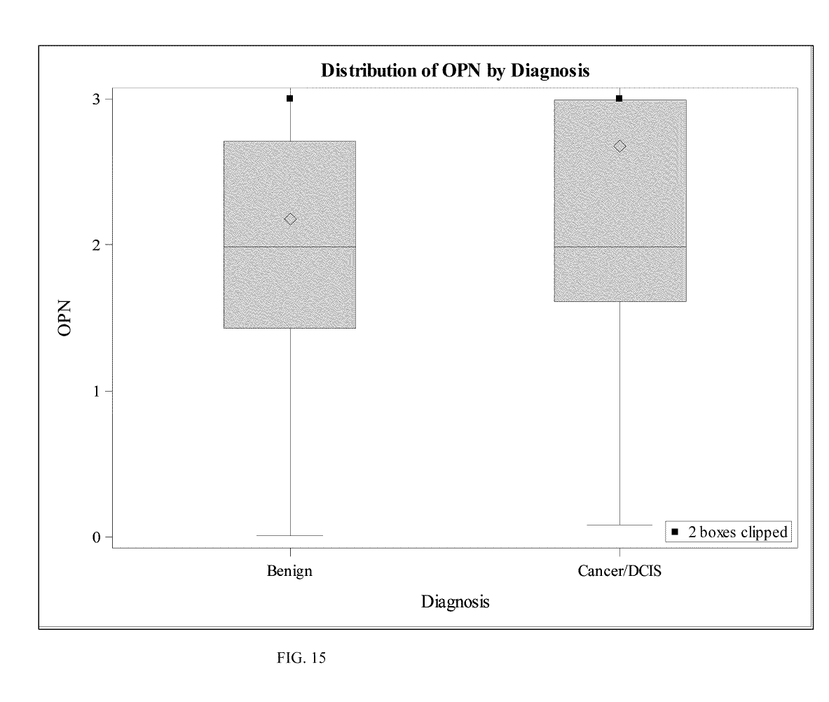

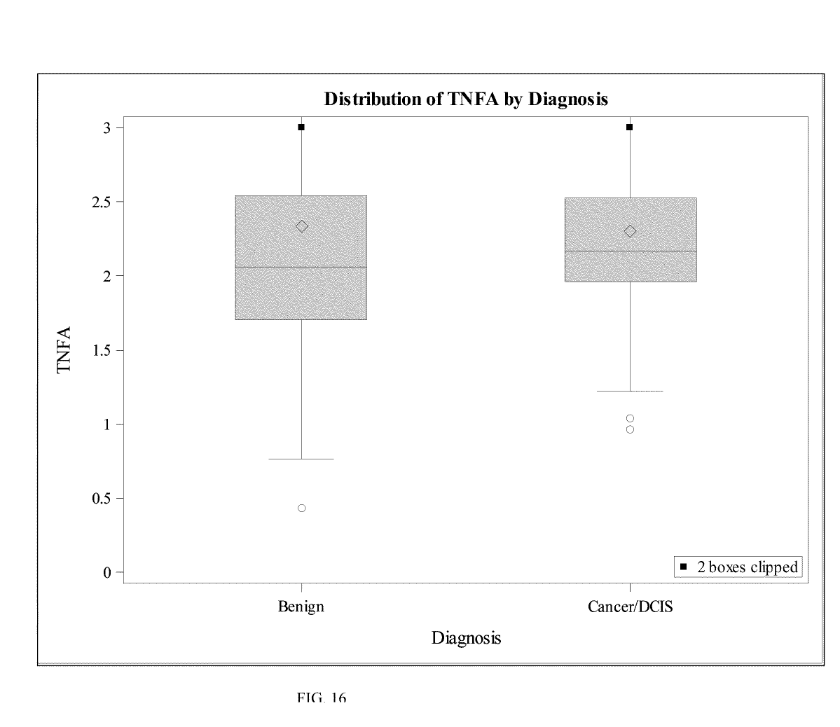

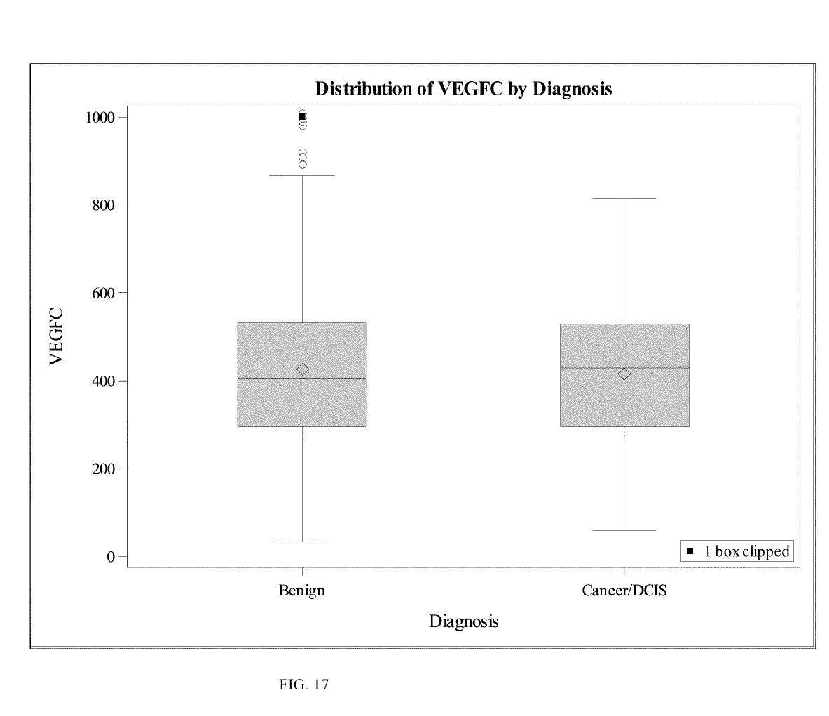

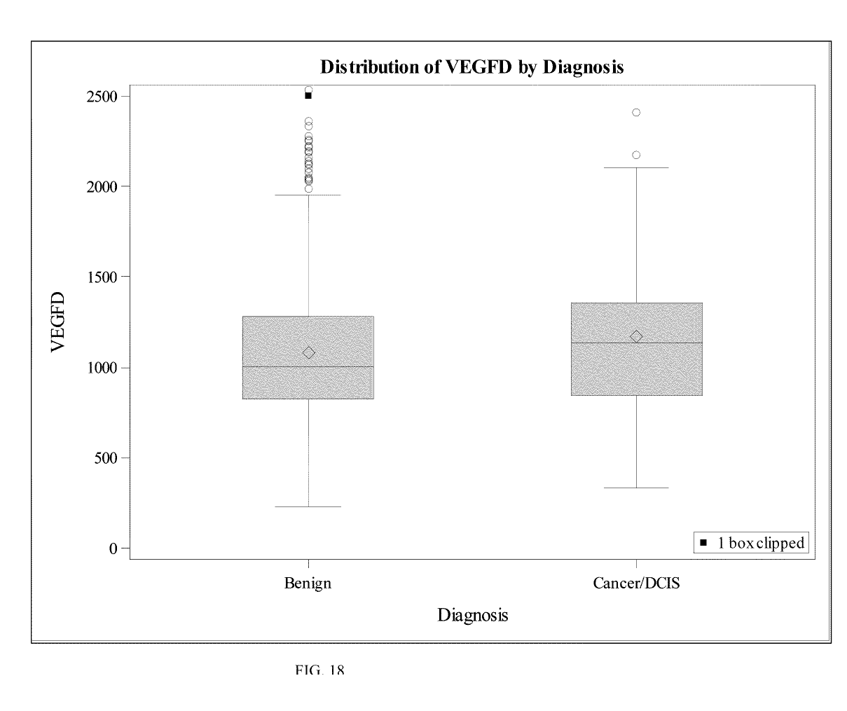

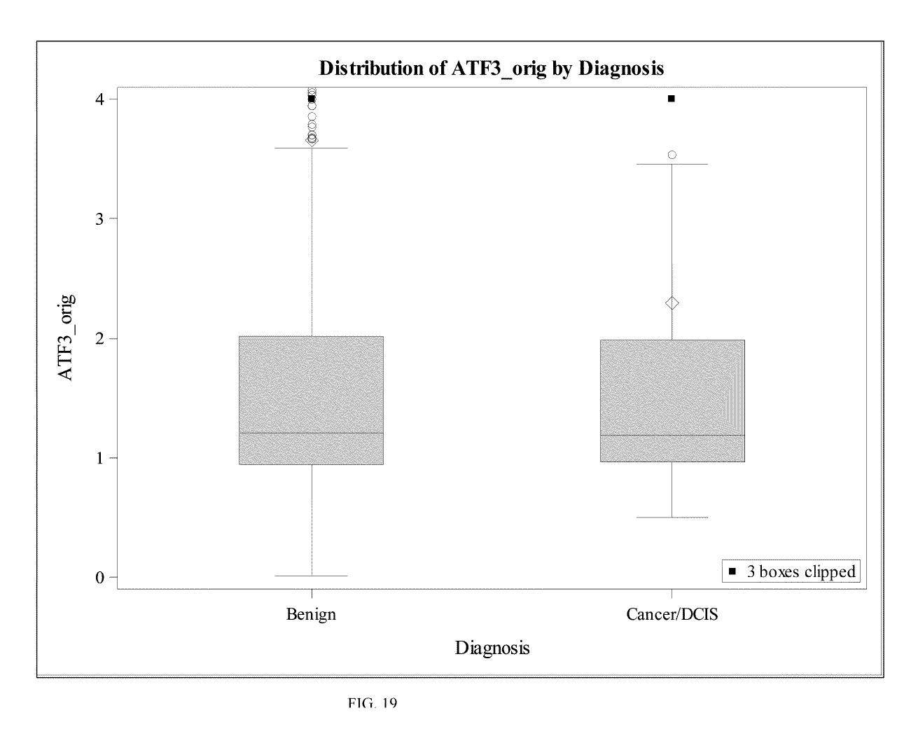

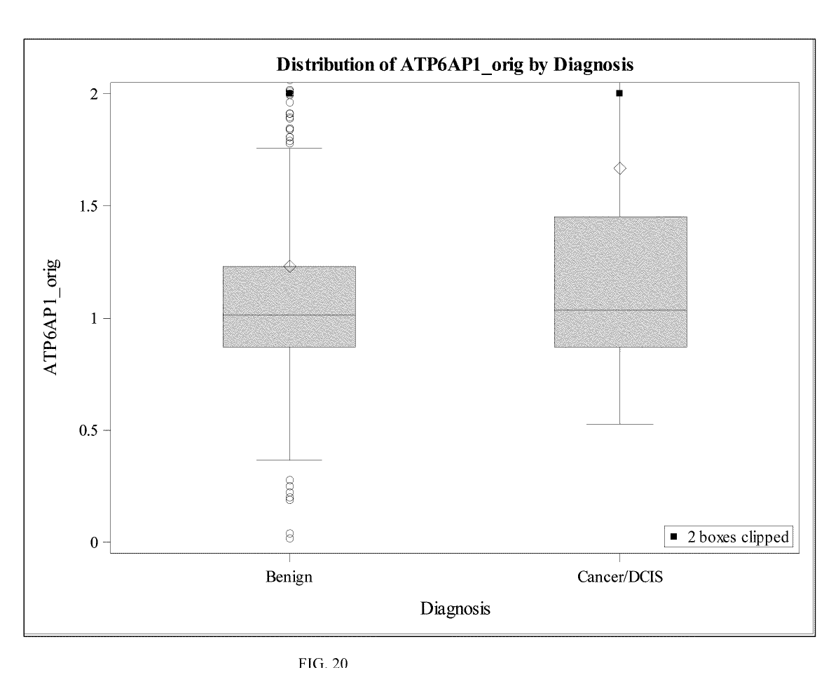

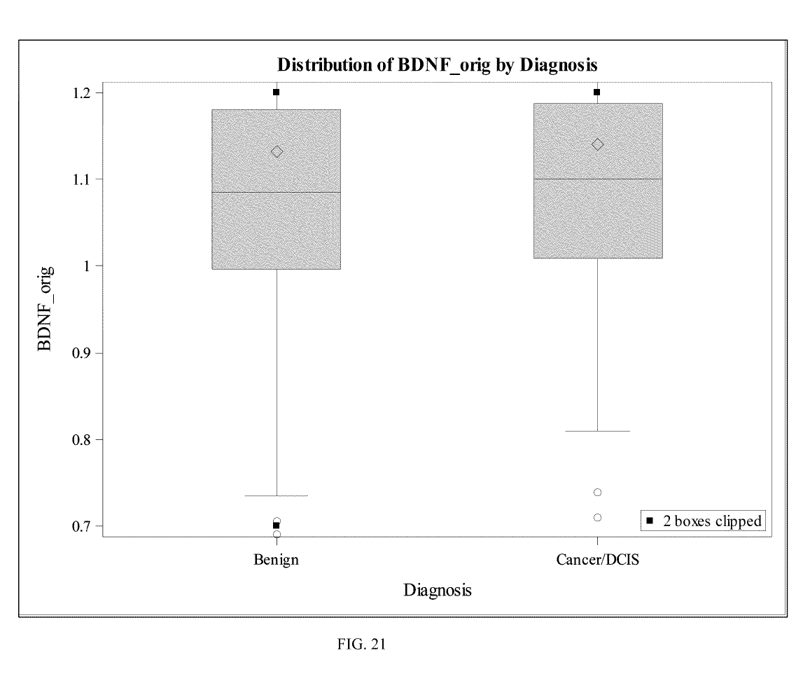

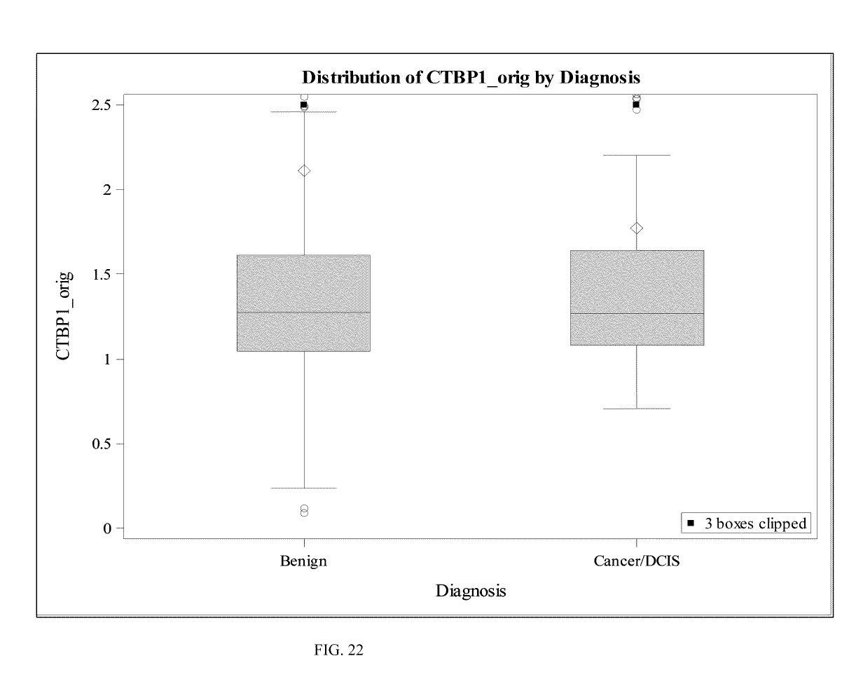

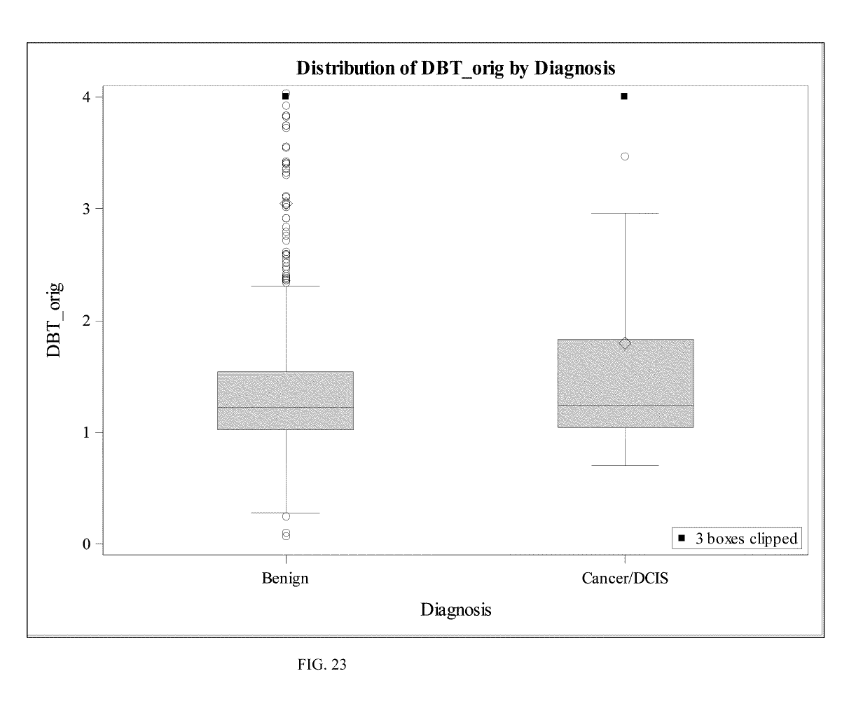

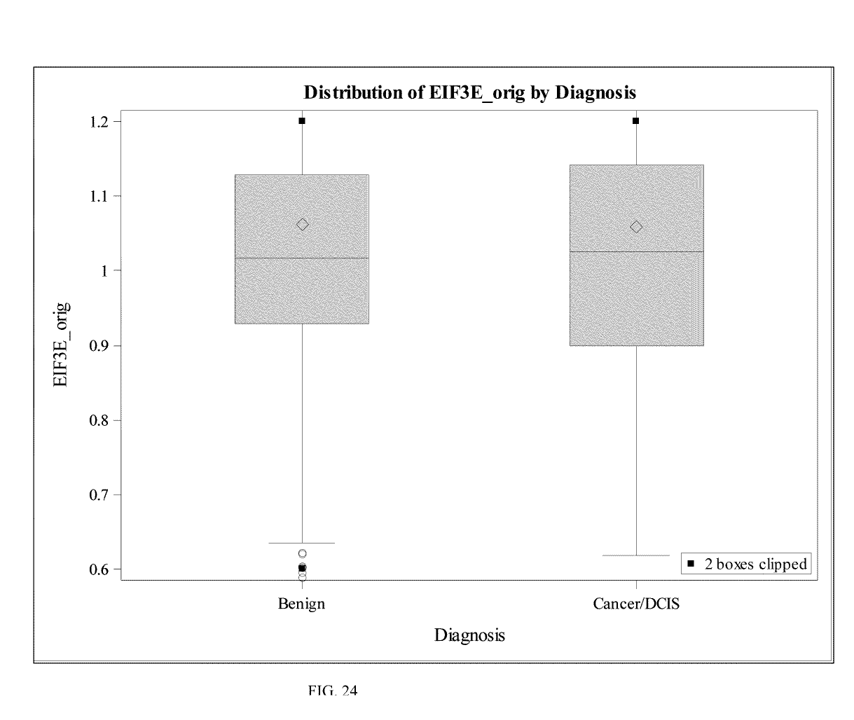

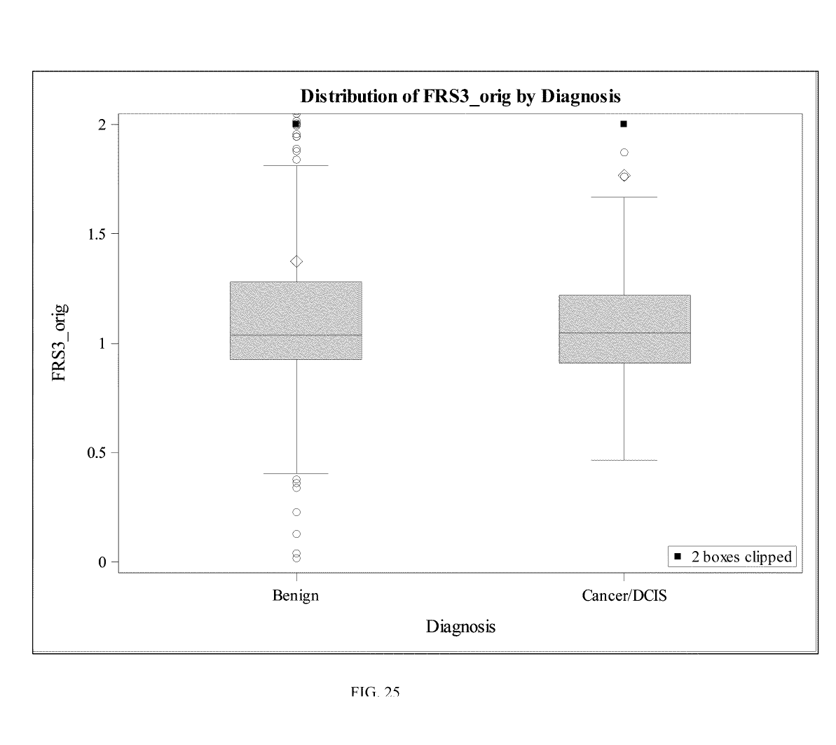

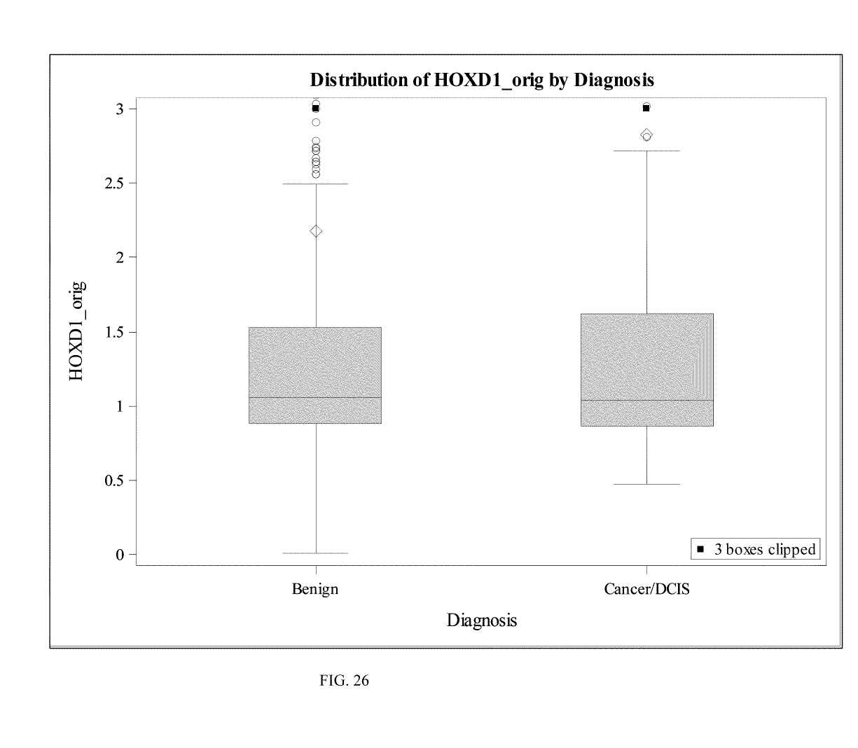

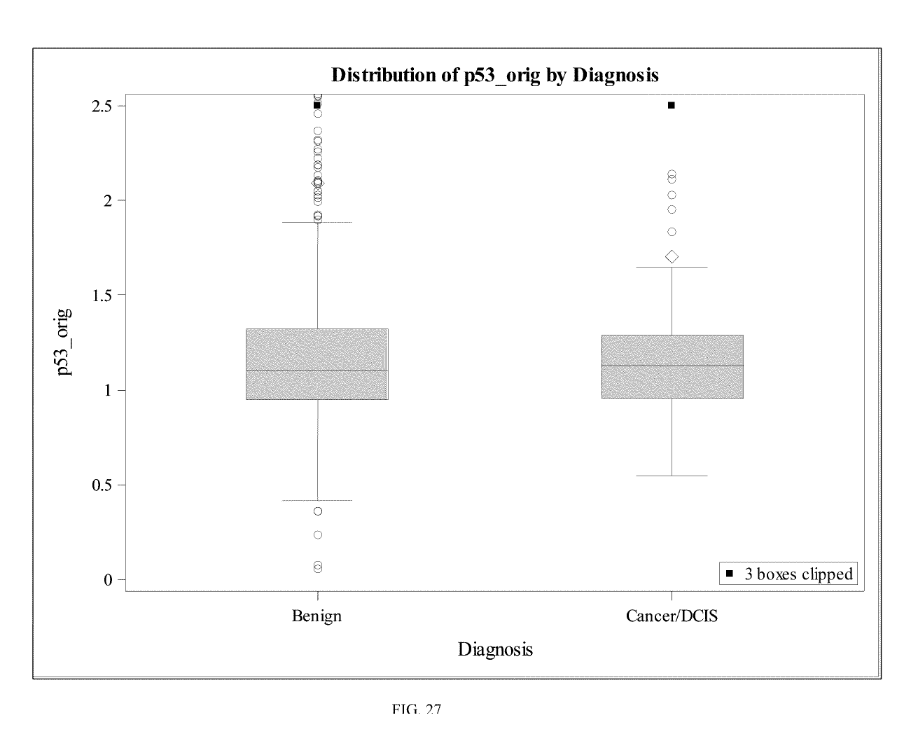

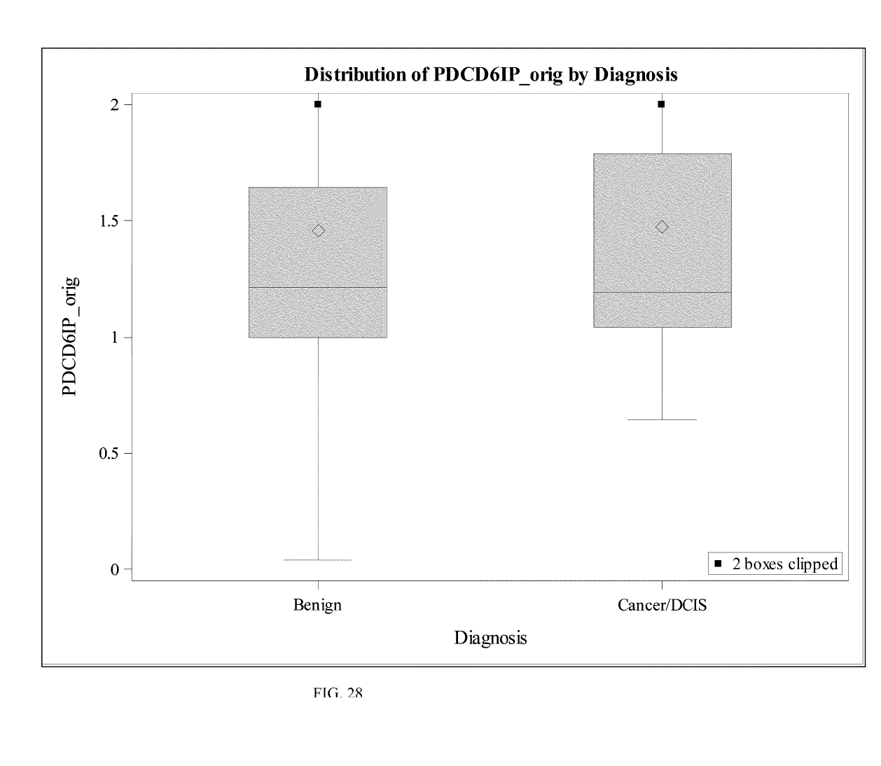

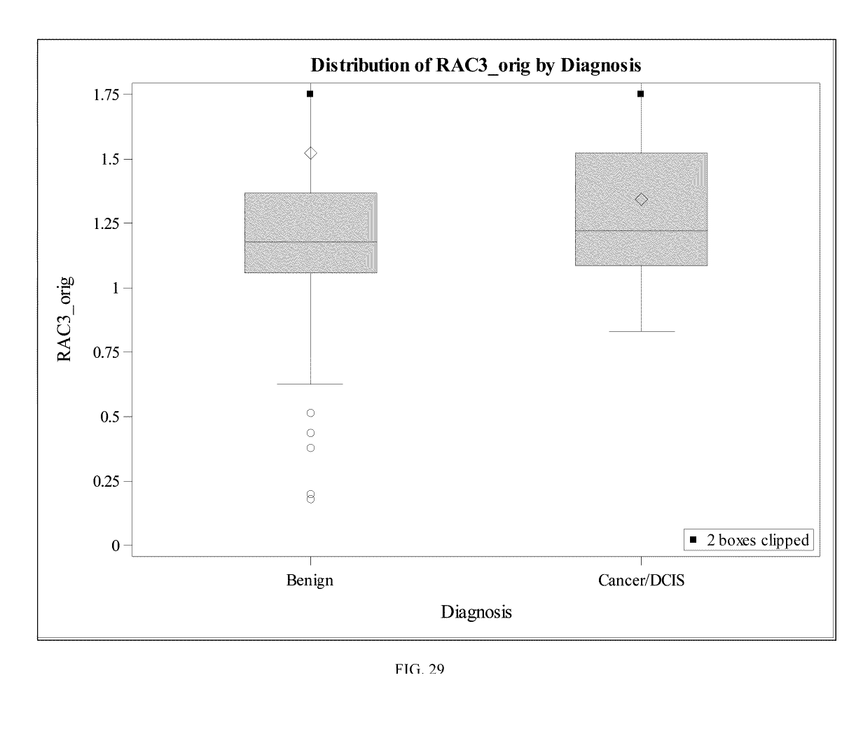

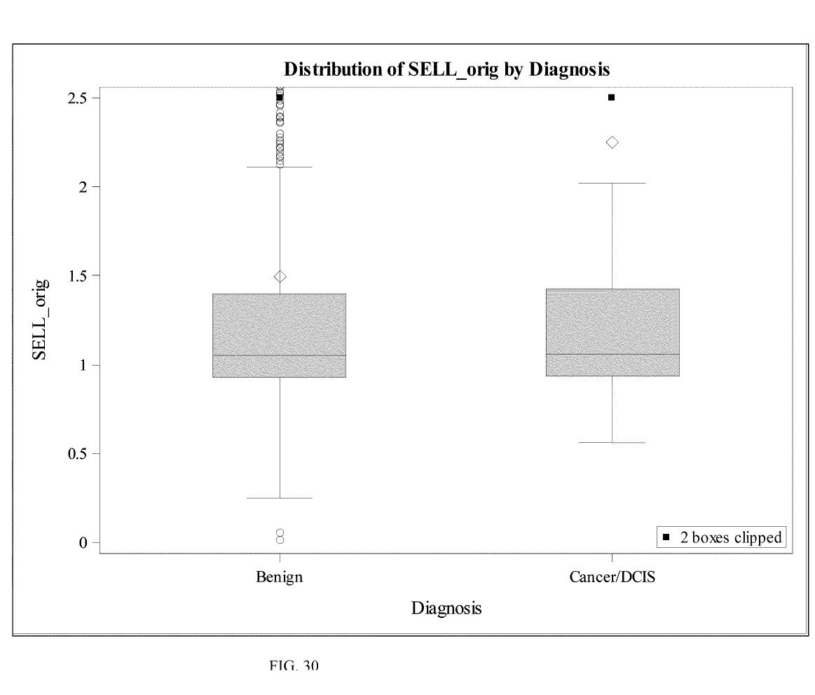

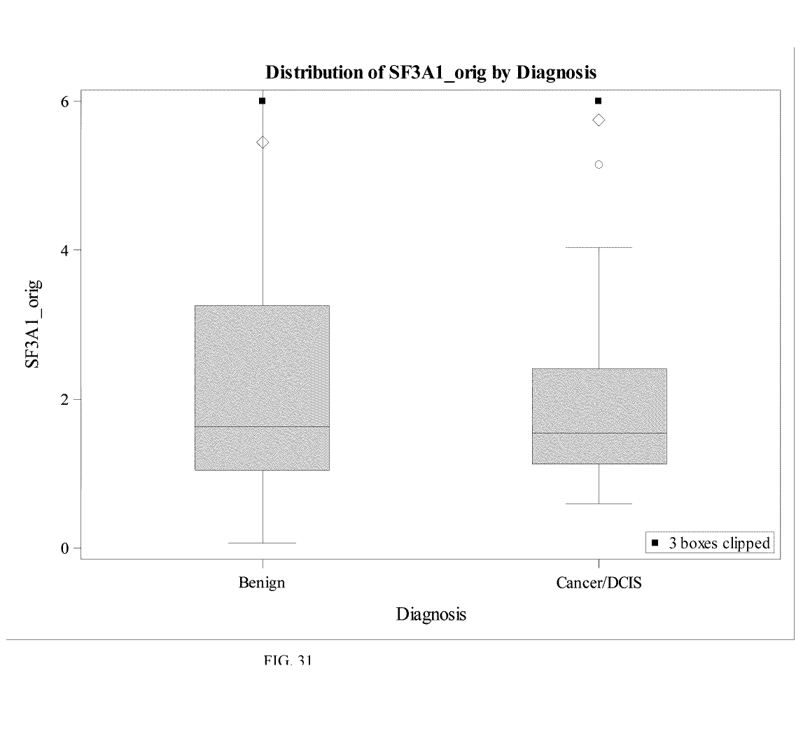

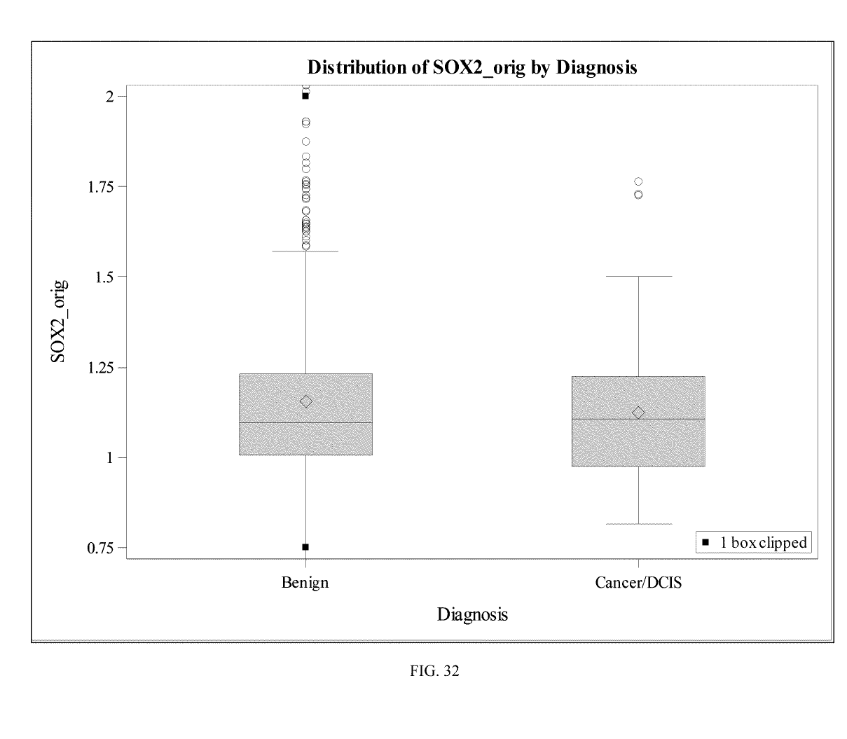

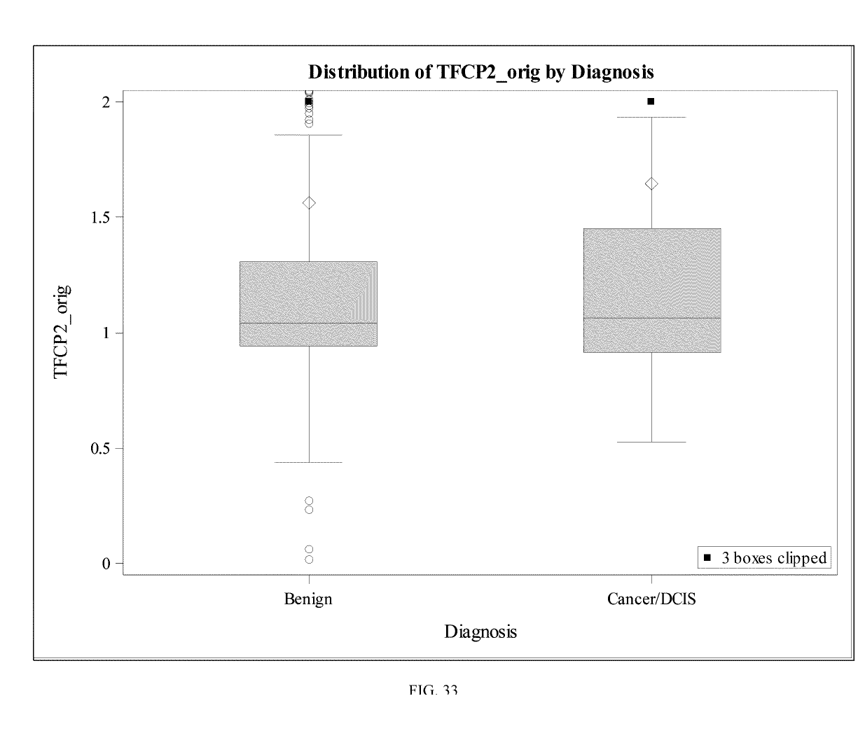

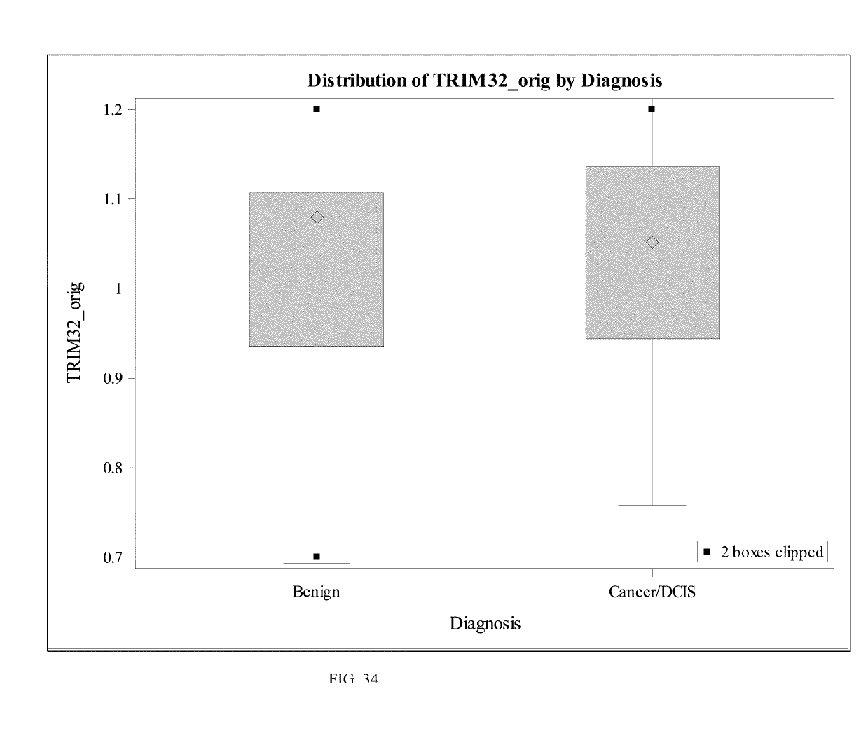

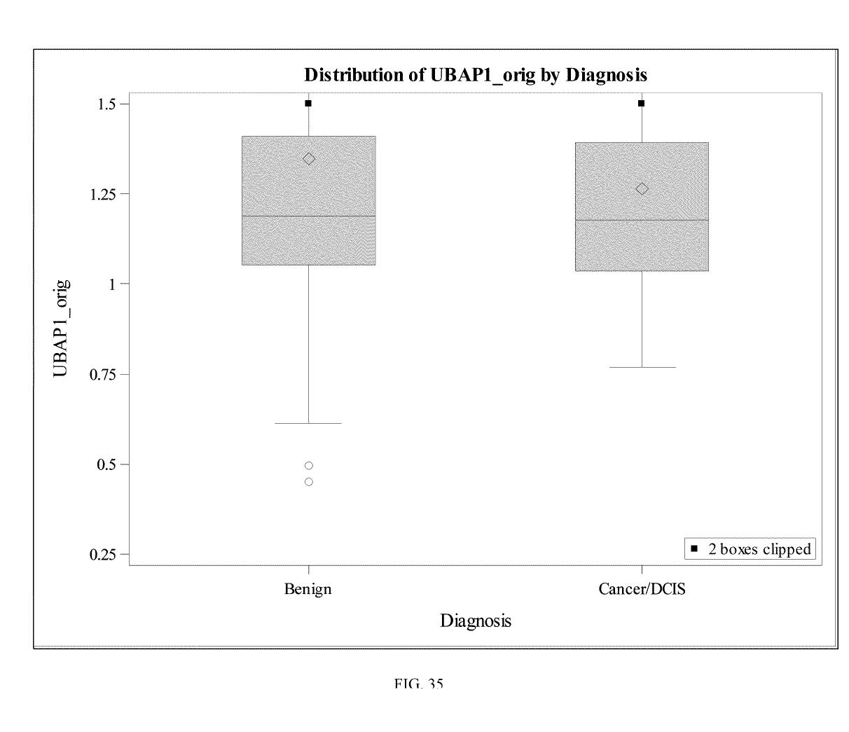

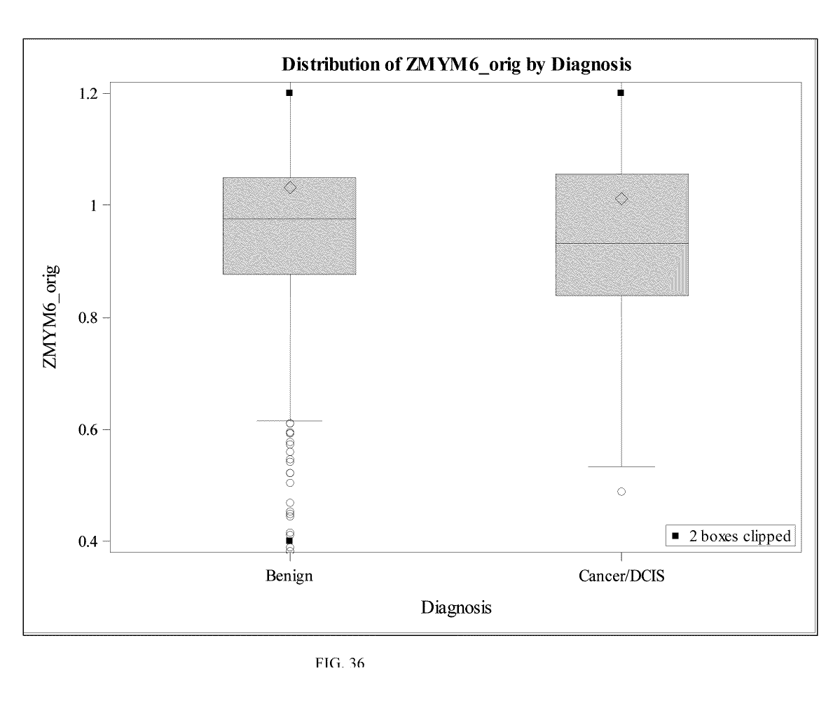

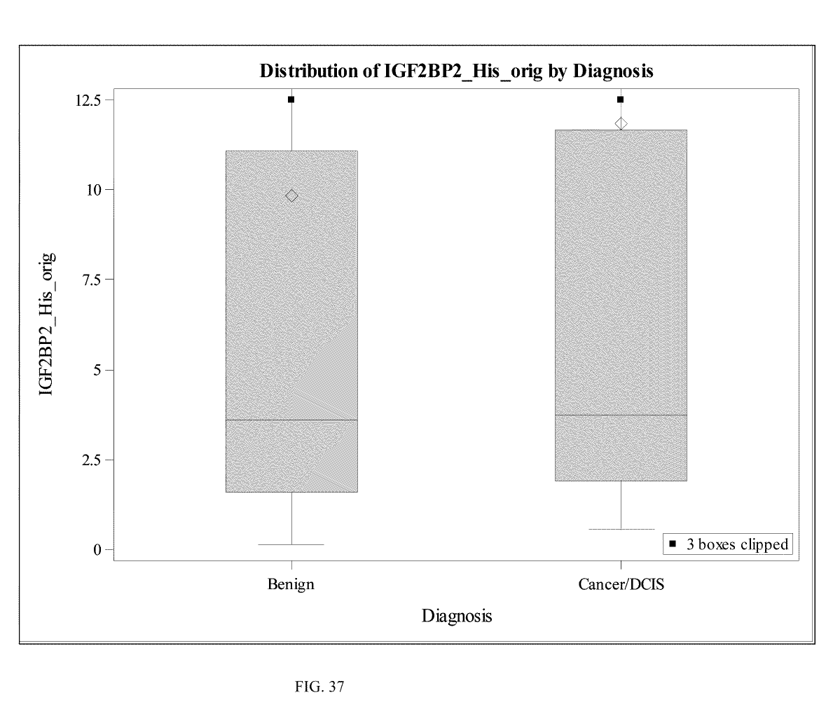

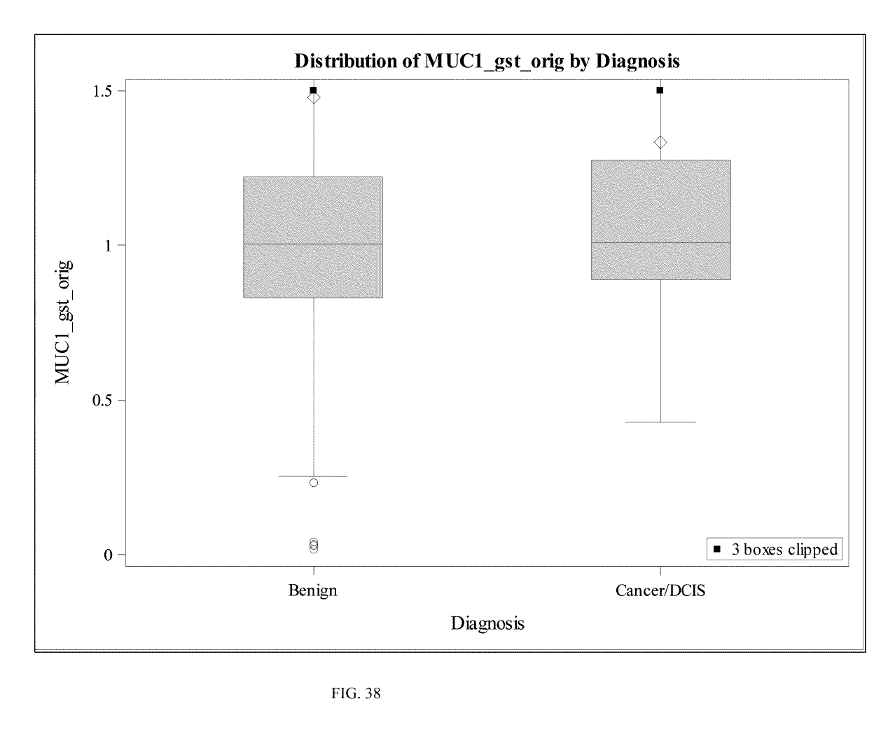

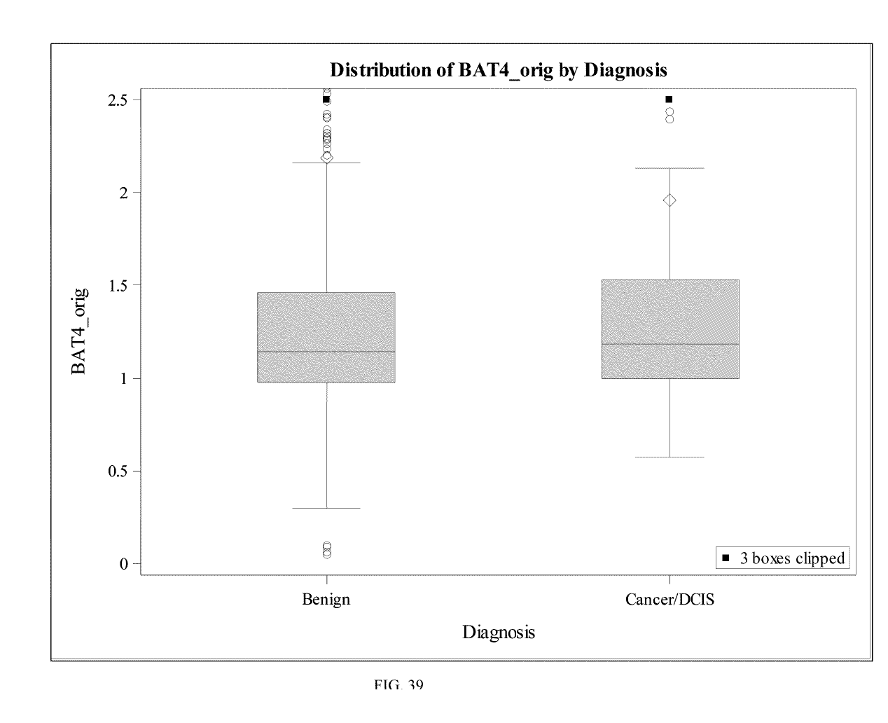

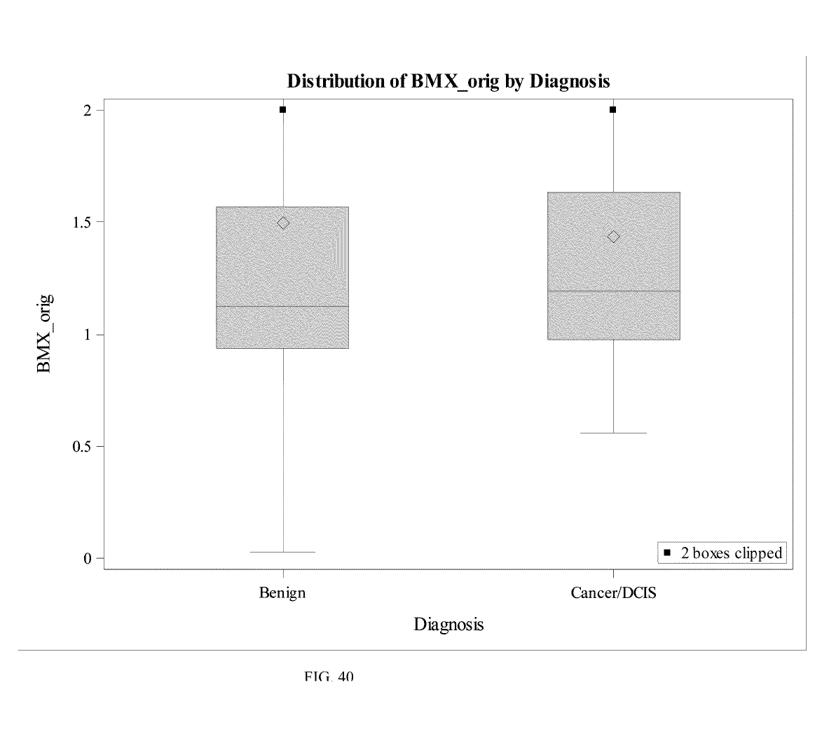

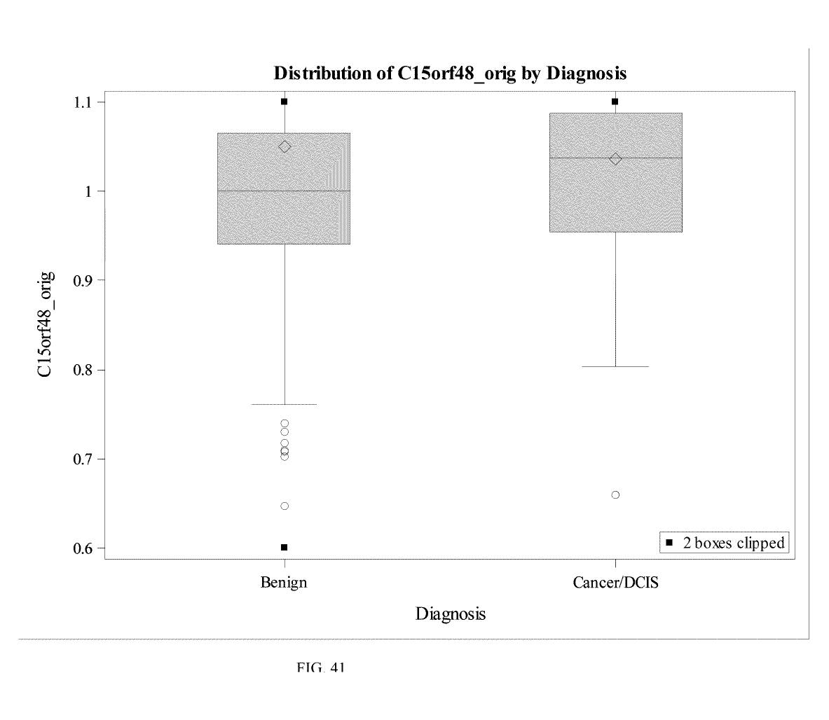

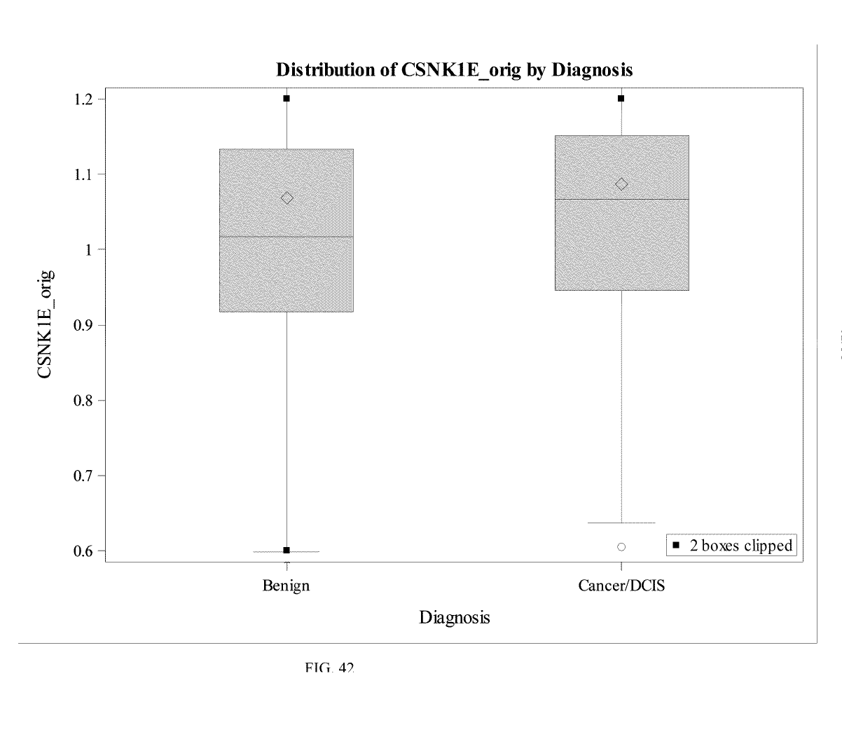

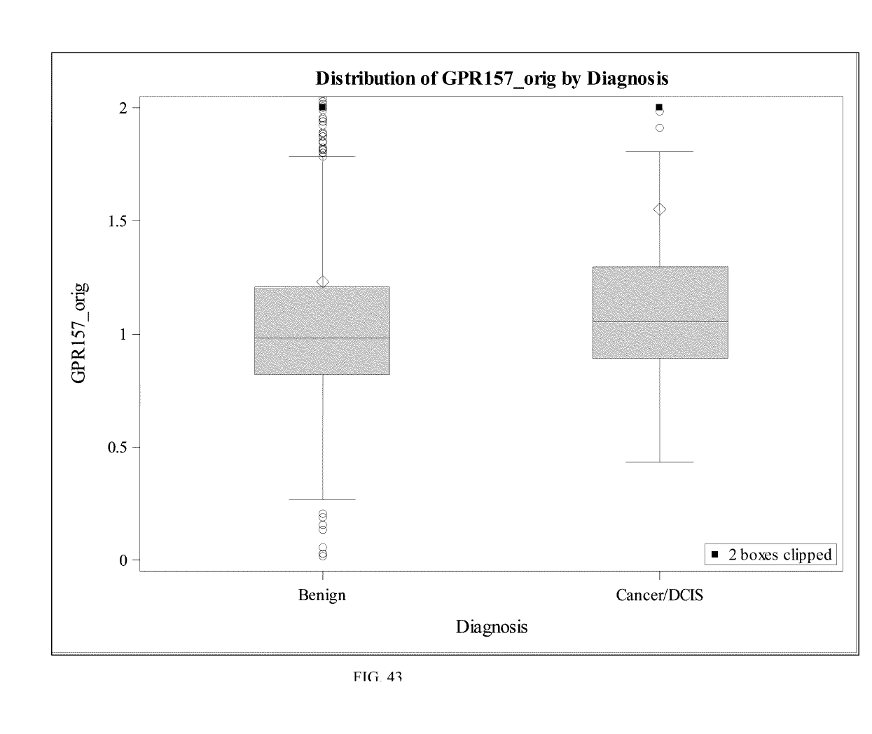

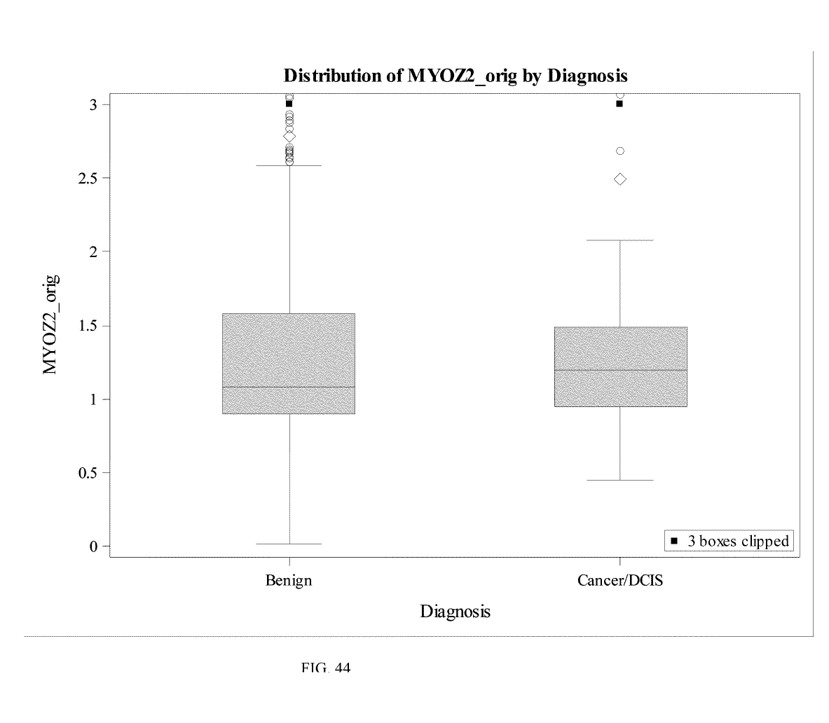

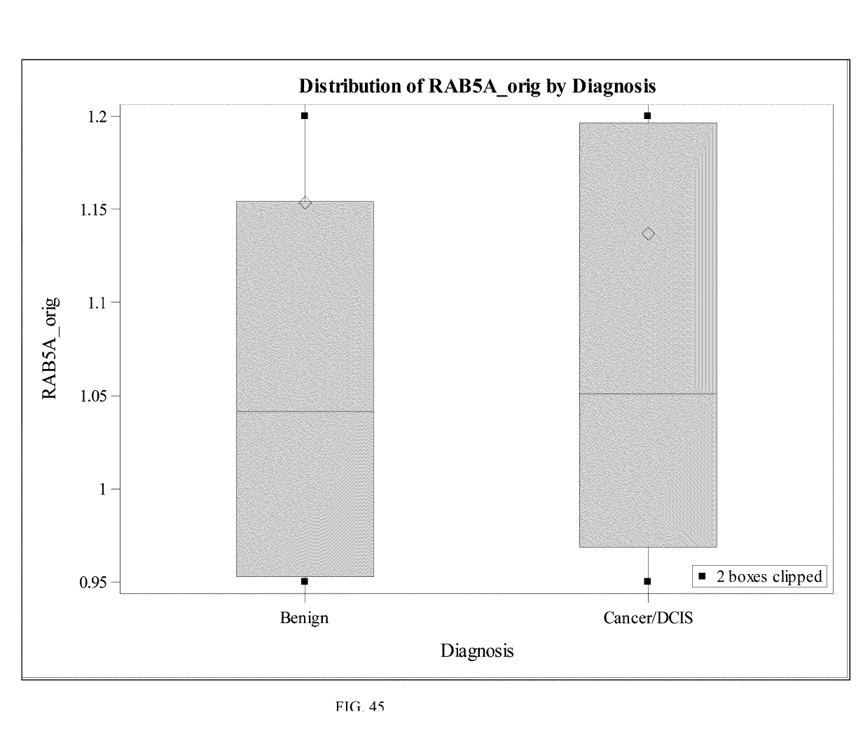

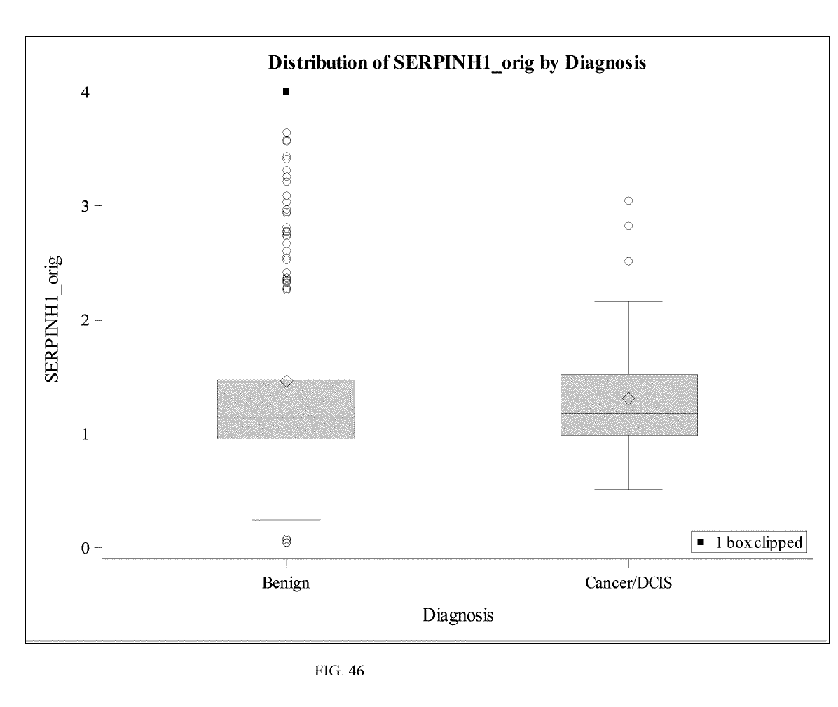

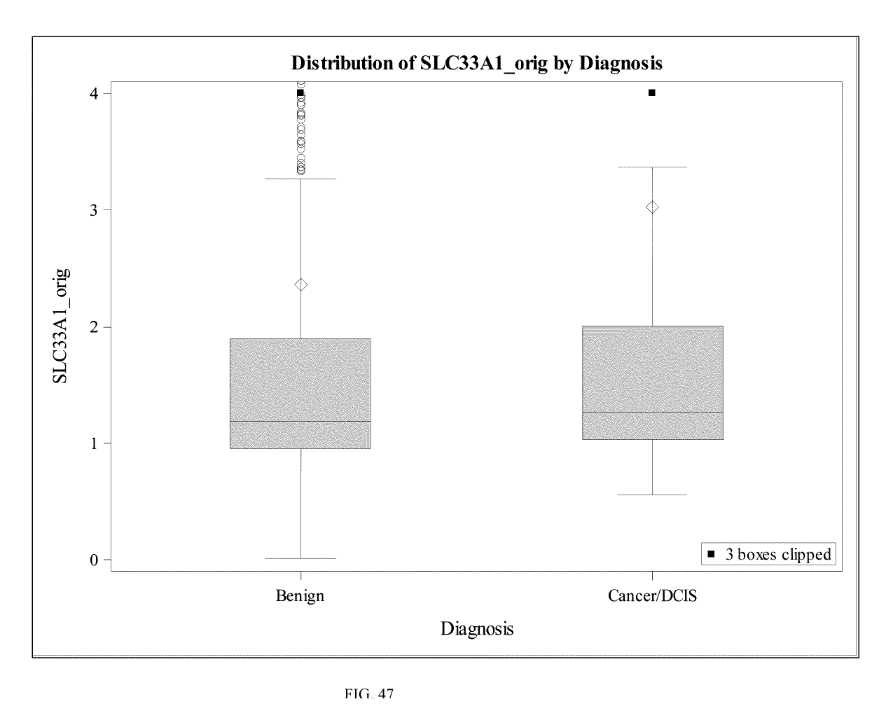

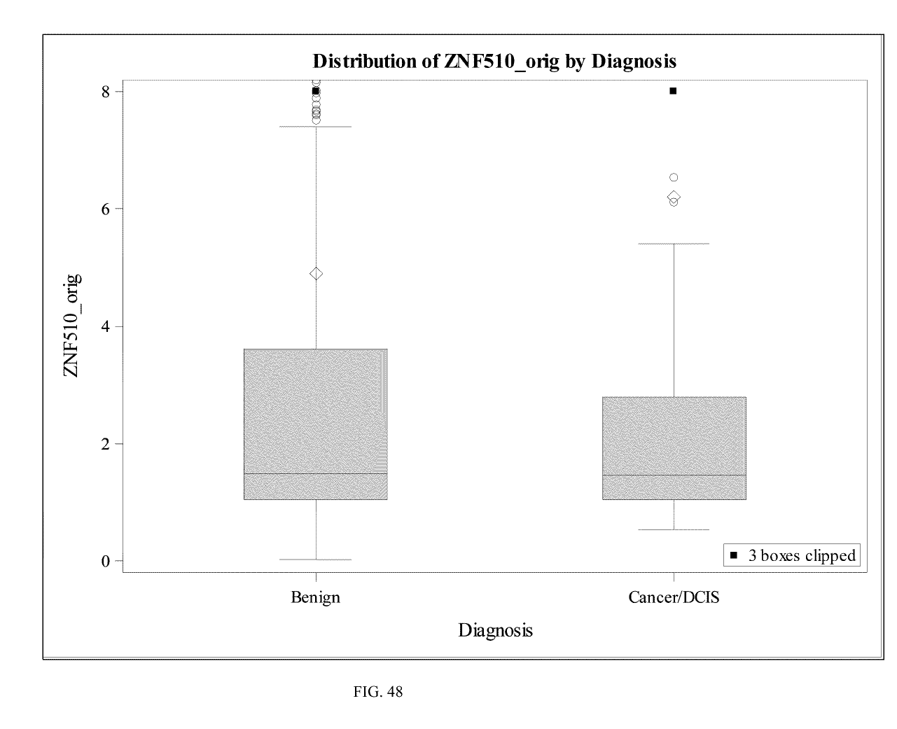

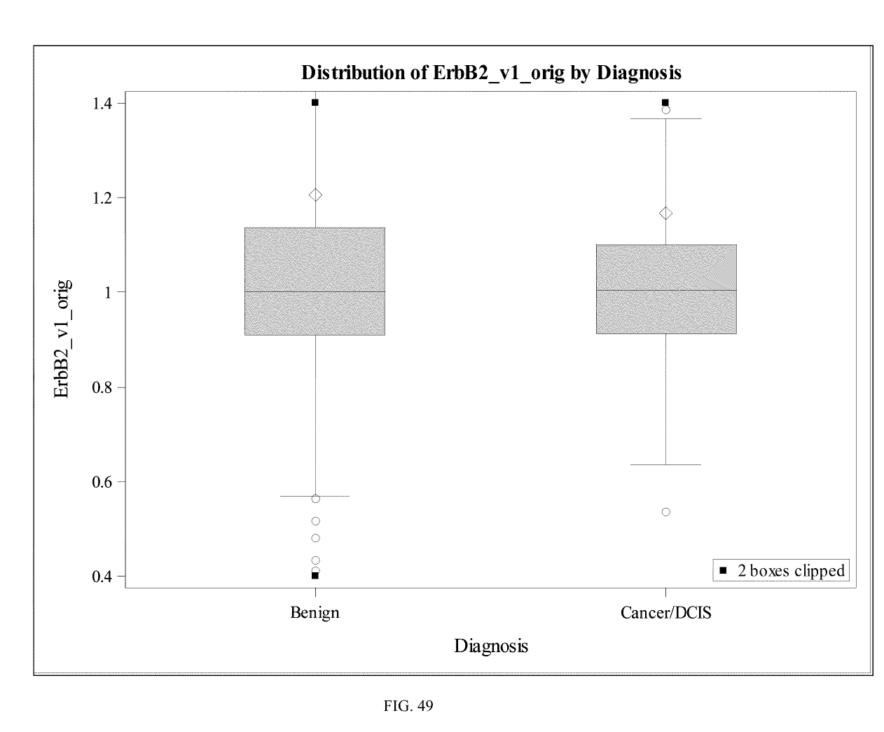

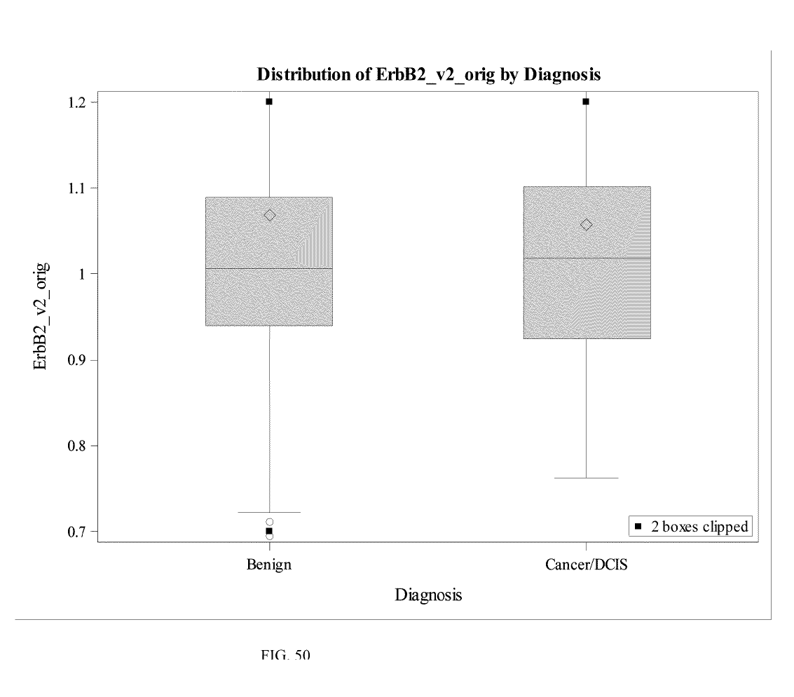

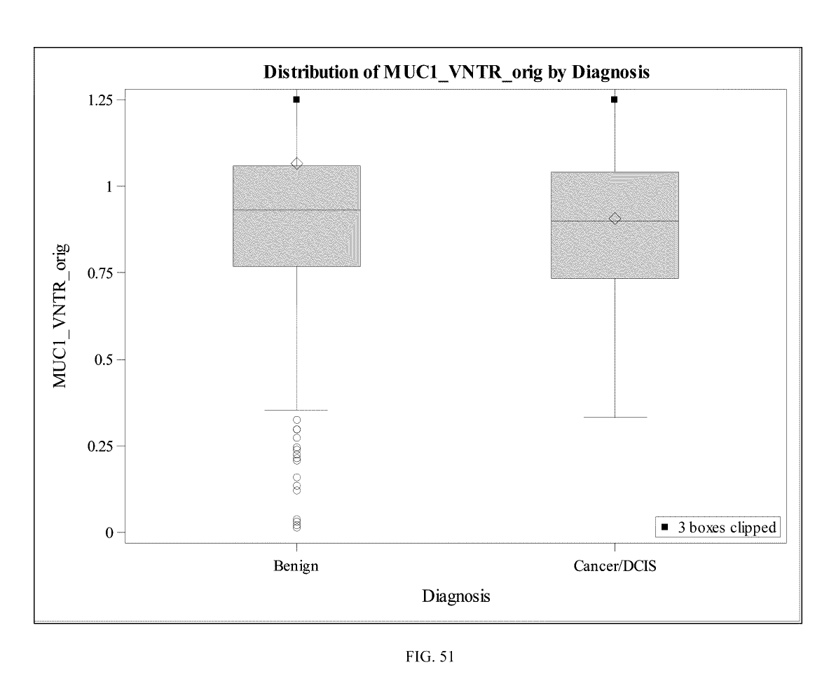

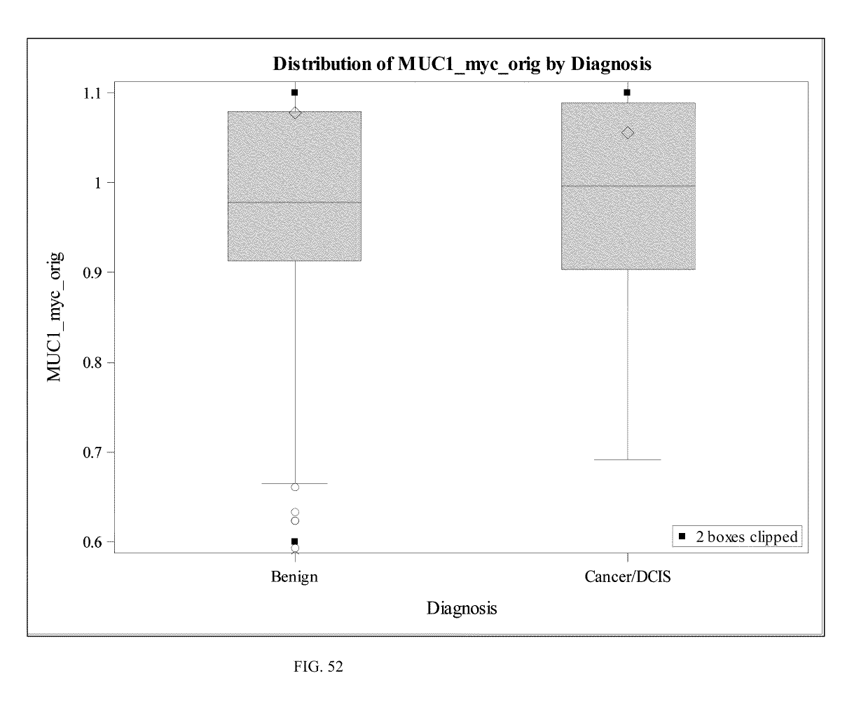

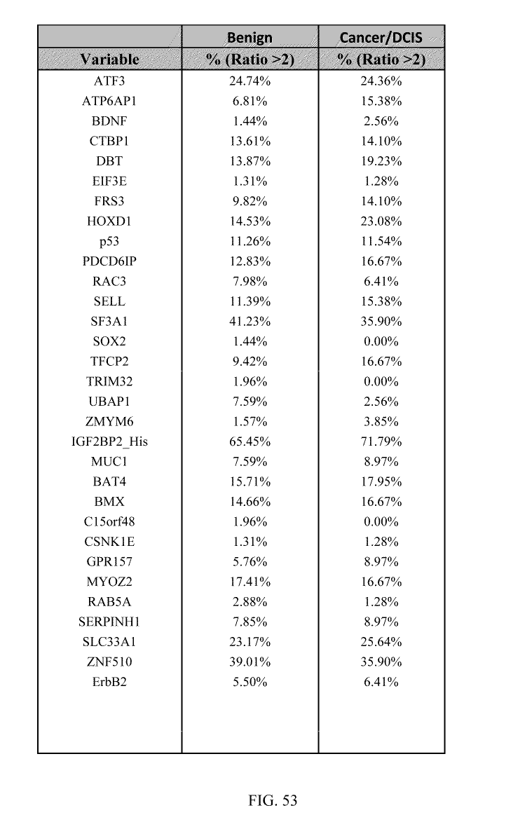

The results shown below are a combination of the population data and the prevalence of tumor types for each population (FIG. 3). The first sub-set population is a group of 163 patients analyzed for SPBs only while the second subset is a group of 31 patients analyzed for both SPBs and AAbs. The prevalence of autoantibody as single markers are shown in both the benign and cancer group (FIG. 5). Of note, the only benign patient who was positive for AAbs in this group had a history of breast cancer. SPB-only and SPB +AAb models have been developed to distinguish benign from invasive lesions (FIGS. 6 and 7).

FIG. 3 shows a table presenting experimental data relating to characteristics of the patient population used to test whether SPBs and AAbs improve prediction of breast cancer. All patients represented were analyzed with 10 SPBs. Subtypes of invasive cancers are also shown.

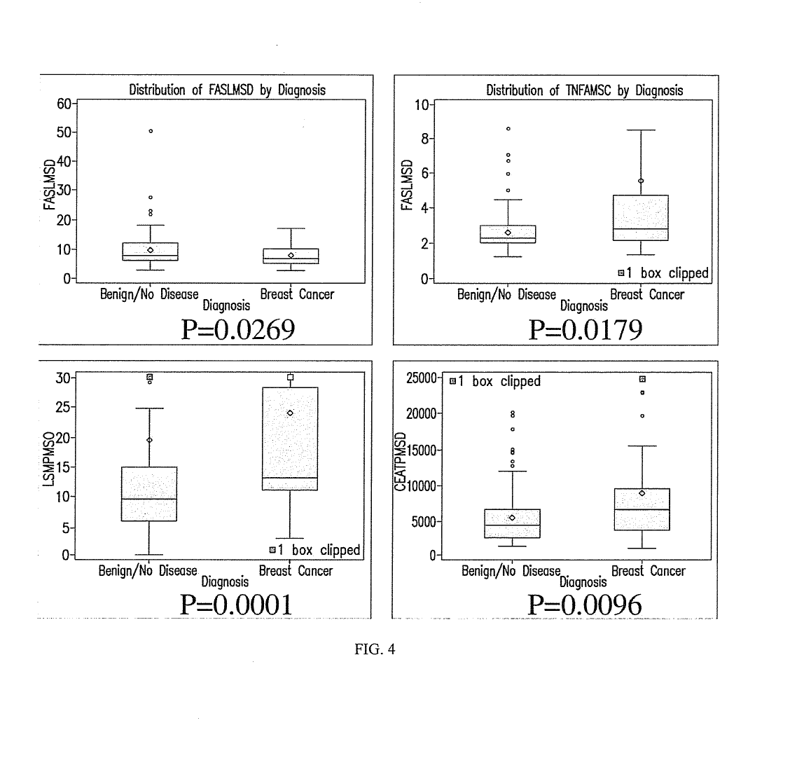

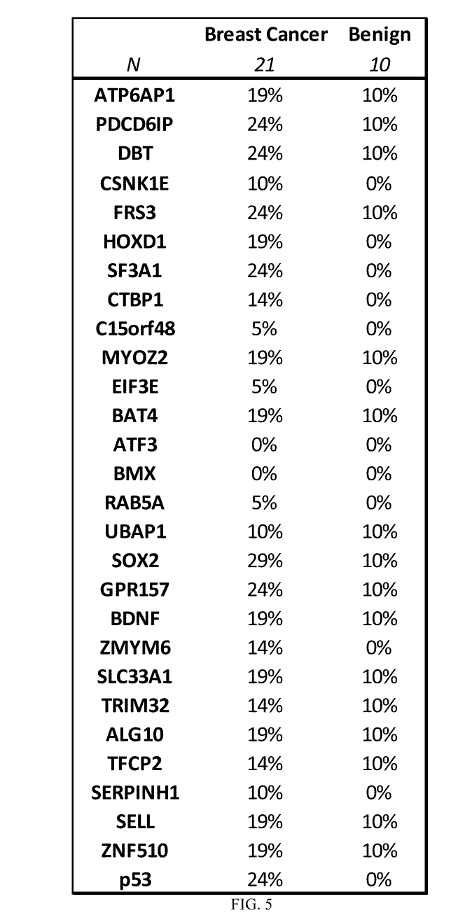

FIG. 4 is a series of graphical representation presenting experimental data. The Figure presents box plots depicting SPB concentrations for selected biomarkers (Upper left=FasL, Upper right=TNF-A, Lower left=IL-8, and Lower right=CEA) in benign and cancer groups in 163 patients. Statistically significant differences are noted. Differences for a single biomarker are significant when comparing means, however, are insufficient to differentiate groups of patients which is why a panel approach is used.

FIG. 5 shows a table listing AAbs used in analysis of contribution to sensitivity/specificity by this class of biomarker. Percentages represent the proportion of patients who were positive for each AAb represented separated by the benign and invasive cases. Note: 100% of the positive signal in benign comes from a patient with a prior breast cancer.

FIG. 6 shows a table presenting experimental data relating to characteristics of the patient population used to test whether SPBs and AAbs improve prediction of breast cancer. All patients represented were analyzed with the combination SPB and AAb panel.

FIG. 7 shows a table of presenting experimental data relating to comparison of models using SPB alone and SPB in combination with AAbs. Shown is the sensitivity, specificity and AUC (Area Under the Curve) which demonstrates a marked increase in each with the addition of AAbs to SPB (n=31).

Conclusions

Using SPBs to distinguishing benign from invasive breast cancer yields reasonable, but non-clinically meaningful results in a combined menopausal population of women (n=163). As hypothesized, the addition of AAbs to a panel of serum protein biomarkers greatly increases sensitivity, specificity and AUC, in a subset of women (n=31). This proof of concept study strongly supports the expansion of a greater number of AAbs in the analysis of women with either benign or cancerous lesions. Multiple randomized prospective trials are underway to establish the combined role of SPB and AAbs to differentiate benign from invasive breast cancers.

### EXAMPLE 2

### Detectio of Breast Cancer

This example demonstrates the proof of concept that AAbs and SPBs combined provide greater sensitivity and specificity to differentiate benign and breast cancer than either biomarker alone.

SPBs in over 351 serum samples from women with either breast cancer or benign breast conditions were measured. SPBs and AAbs associated with breast cancer were analyzed in these patients.

Methods and Population

351 serum samples were obtained for a prospectively collected set of patients who were being evaluated for a suspicious mass. A SPB training model using MSD based instrumentation was developed to determine protein concentrations and develop predictive ranges for women with benign and invasive lesions.

Results

The results shown below Tables 1 and 2 are a combination of the population data and the prevalence of tumor types for each population.

TABLE 1

| | Training | | Validation | | Total |
|---|---|---|---|---|---|
| N | 200 | | 151 | | 351 |
| Race | | | | | |
| White | 159 | 80% | 120 | 79% | 279 |
| Black/African American | 10 | 5% | 9 | 6% | 19 |
| Asian | 8 | 4% | 6 | 4% | 14 |
| Hispanic | 20 | 10% | 11 | 7% | 31 |
| Other | 2 | 1% | 5 | 3% | 7 |
| Unknown | 1 | 1% | 0 | 0% | 1 |

TABLE 2

| | Total |
|---|---|
| N | 351 |
| BIRAD | |
| 3 | 143 |
| 4 | 82 |
| 4A | 96 |
| 4B | 21 |
| 4C | 9 |
| Biopsies | 197 |
| Benign and | 166 |
| Presumed | |
| Carcinoma in situ | |
| ductal | 10 |
| lobular | 2 |
| Invasive carcinoma * | 19 |

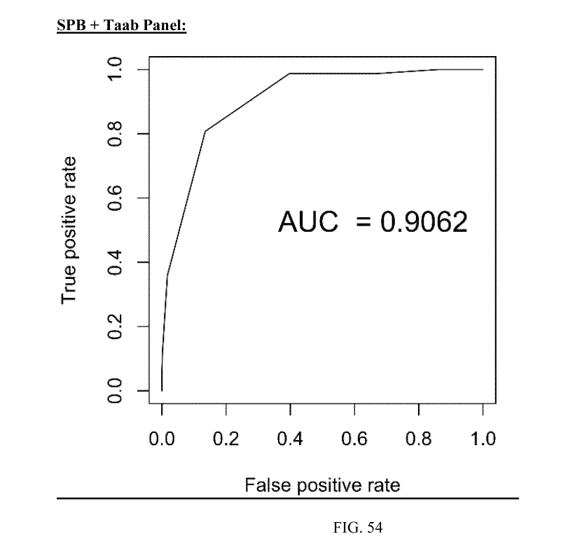

The population is a group of 351 patients analyzed for SPBs, AAbs only and both SPBs and AAbs. FIG. 53 shows a table listing AAbs used in analysis of contribution to sensitivity/specificity by this class of biomarker. Percentages represent the proportion of patients who were positive for each AAb represented separated by the benign and invasive cases.

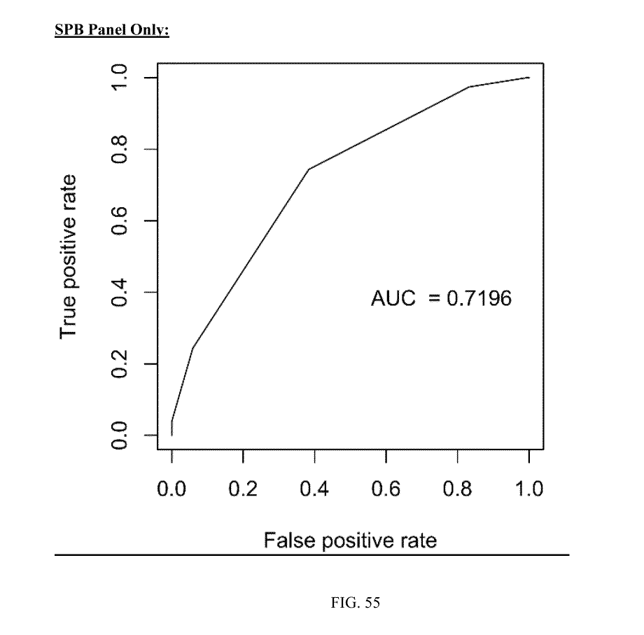

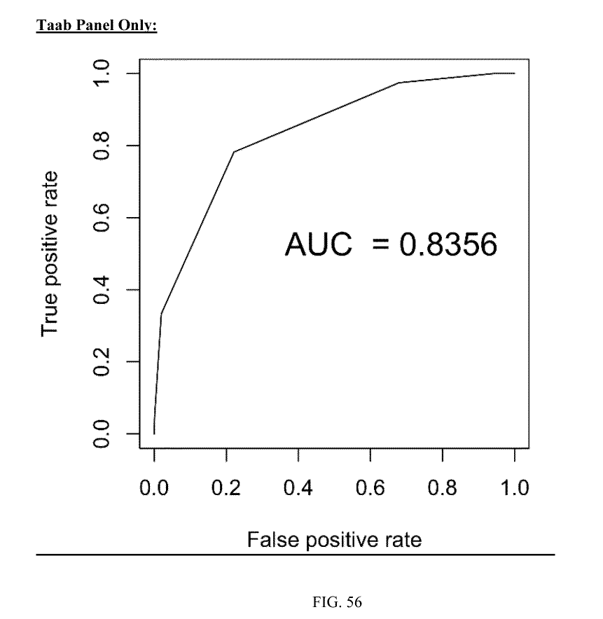

FIG. 54 shows sensitivity and specificity of detection utilizing SPBs in combination with AAbs (as AUC (Area Under the Curve), while FIG. 55 shows sensitivity and specificity of detection utilizing SPBs alone and FIG. 56 shows sensitivity and specificity of detection utilizing AAbs alone.

Conclusions

Using SPBs to distinguishing benign from invasive breast cancer yields reasonable, but non-clinically meaningful results in a combined population of women. As hypothesized, the addition of AAbs to a panel of serum protein biomarkers greatly increases sensitivity, specificity and AUC. This proof of concept study strongly supports the expansion of a greater number of AAbs in the analysis of women with either benign or cancerous lesions. Multiple randomized prospective trials are underway to establish the combined role of SPB and AAbs to differentiate benign from invasive breast cancers.

Although the invention has been described with reference to the above example, it will be understood that modifications and variations are encompassed within the spirit and scope of the invention. Accordingly, the invention is limited only by the following claims.

What is claimed is:

1. A method for measuring the level of a protein biomarker and an autoantibody in a sample from a subject having or at risk of having breast cancer comprising:
a) obtaining a biological sample from the subject; and
b) measuring a level of at least one protein biomarker and at least one autoantibody, wherein the at least one protein biomarker is selected from FasL, TNF-A, IL-8, and CEA.

2. The method of claim 1, wherein the sample is a bodily fluid such as ascites, serum, plasma, feces, lymph, cerebrospinal fluid, nipple aspirate, or urine.

3. The method of claim 1, wherein the at least one protein biomarker further comprises one or more of ERBB2, HGF, IFNG, IL6, OPN, VEGFC, VEGFD, ATF3, ATP6AP1, BDNF, CTBP1, DBT, EIF3E, FRS3, HOXD1, p53, PDCD6IP, RAC3, SELL, SF3A1, SOX2, TFCP2, TRIMP2, UBAP1, ZMYM6, IGF2PB2, MUC1, BAT4, BMX, C15orf48, CSNK1E, GPR157, MYOZ2, RAB5A, SERPINH1, SLC33A1 and ZNF510.

4. The method of claim 1, wherein the autoantibody specifically binds RAC3, IGF2BP2, MUC1, ErbB2, ATP6AP1, PDCD6IP, DBT, CSNK1E, FRS3, HOXD1, SF3A1, CTBP1, C15orf48, MYOZ2, EIF3E, BAT4, ATF3, BMX, RAB5A, UBAP1, SOX2, GPR157, BDNF, ZMYM6, SLC33A1, TRIM32, ALG10, TFCP2, SERPINH1, SELL, ZNF510 or p53.

5. The method of claim 1, wherein the autoantibody specifically binds p53 and the biomarker is at least one of FasL, TNF-A, IL-8, and CEA, or any combination thereof.

6. The method of claim 1, wherein the method further comprises histological analysis of a biopsy tissue.

7. The method of claim 1, wherein the method further comprises image analysis.

8. The method of claim 1, wherein the level of the at least one protein biomarker is determined via protein array analysis.

9. The method of claim 1, wherein the subject is a mammal.

10. The method of claim 1, wherein the mammal is a human.

11. The method of claim 1, further comprising administering the subject a therapeutic agent.

12. The method of claim 1, further comprising prescribing the patient a therapeutic regime.

13. The method of claim 1, wherein (b) comprises measuring an expression product of the at least one protein biomarker or the at least one autoantibody.

14. The method of claim 13, wherein the expression product is protein, microRNA or mRNA.

15. A kit for detecting breast cancer in a subject, comprising means for detecting in a biological sample at least one protein biomarker and at least one autoantibody, the at least one protein biomarker being a genomic sequence, transcript, or protein of one or more of FasL, TNF-A, IL-8, and CEA.

16. The kit of claim 15, further comprising a container suitable for containing the means and the biological sample.

17. The kit of claim 15, wherein the kit comprises a nucleic acid probe specific for the biomarker or autoantibody.

18. The kit of claim 15, wherein the kit comprises a pair of primers for specific amplification of a transcript of the biomarker or autoantibody.

19. The kit of claim 15, wherein the kit comprises an antibody specific for a biomarker or autoantibody.

20. The kit of claim 15, wherein protein, microRNA or mRNA is measured.

21. The kit of claim 15, wherein the autoantibody specifically binds RAC3, IGF2BP2, MUC1, ErbB2, ATP6AP1, PDCD6IP, DBT, CSNK1E, FRS3, HOXD1, SF3A1, CTBP1, C15orf48, MYOZ2, EIF3E, BAT4, ATF3, BMX, RAB5A, UBAP1, SOX2, GPR157, BDNF, ZMYM6, SLC33A1, TRIM32, ALG10, TFCP2, SERPINH1, SELL, ZNF510 or p53.

22. The kit of claim 15, further comprising a genomic sequence, transcript, or protein of one or more of ERBB2, HGF, IFNG, IL6, OPN, VEGFC, VEGFD, ATF3, ATP6AP1, BDNF, CTBP1, DBT, EIF3E, FRS3, HOXD1, p53, PDCD6IP, RAC3, SELL, SF3A1, SOX2, TFCP2, TRIMP2, UBAP1, ZMYM6, IGF2PB2, MUC1, BAT4, BMX, C15orf48, CSNK1E, GPR157, MYOZ2, RAB5A, SERPINH1, SLC33A1 and ZNF510.

23. An array comprising a plurality of probes for specifically binding a biomarker and an autoantibody, wherein the biomarker is at least one or more of FasL, TNF-A, IL-8, and CEA.

24. The array of claim 23, wherein the plurality of probes are oligonucleotides.

25. The array of claim 23, wherein the plurality of probes are polypeptides.

26. The array of claim 25, wherein the plurality of probes are antibodies.

27. The array of claim 23, wherein the autoantibody specifically binds RAC3, IGF2BP2, MUC1, ErbB2, ATP6AP1, PDCD6IP, DBT, CSNK1E, FRS3, HOXD1, SF3A1, CTBP1, C15orf48, MYOZ2, EIF3E, BAT4, ATF3, BMX, RAB5A, UBAP1, SOX2, GPR157, BDNF, ZMYM6, SLC33A1, TRIM32, ALG10, TFCP2, SERPINH1, SELL, ZNF510 or ₚ53.

28. The array of claim 23, wherein the biomarker further comprises one or more of ERBB2, HGF, IFNG, IL6, OPN, VEGFC, VEGFD, ATF3, ATP6AP1, BDNF, CTBP1, DBT, EIF3E, FRS3, HOXD1, p53, PDCD6IP, RAC3, SELL, SF3A1, SOX2, TFCP2, TRIMP2, UBAP1, ZMYM6, IGF2PB2, MUC1, BAT4, BMX, C15orf48, CSNK1E, GPR157, MYOZ2, RAB5A, SERPINH1, SLC33A1 and ZNF510.

29. A panel for detecting breast cancer in a subject, the panel comprising:
(a) one or more of the following biomarker proteins or fragments thereof: FasL, TNFA, IL8, CEA, ERBB2, HGF, IFNG, IL6, OPN, VEGFC, VEGFD, ATF3, ATP6AP1, BDNF, CTBP1, DBT, EIF3E, FRS3, HOXD1, p53, PDCD6IP, RAC3, SELL, SF3A1, SOX2, TFCP2, TRIMP2, UBAP1, ZMYM6, IGF2PB2, MUC1, BAT4, BMX, C15orf48, CSNK1E, GPR157, MYOZ2, RAB5A, SERPINH1, SLC33A1 and ZNF510; and
(b) one or more autoantibodies that specifically bind one or more of RAC3, IGF2BP2, MUC1, ErbB 2, ATP6AP1, PDCD6IP, DBT, CSNK1E, FRS3, HOXD1, SF3A1, CTBP1, C15orf48, MYOZ2, EIF3E, BAT4, ATF3, BMX, RAB5A, UBAP1, SOX2, GPR157, BDNF, ZMYM6, SLC33A1, TRIM32, ALG10, TFCP2, SERPINH1, SELL, ZNF510 or p53.
